SEQUENCE LISTING (10 sequences)

SEQ ID NO: 1 - DNA, 133 bp, Listeria monocytogenes
ataaagcaagcatataatattgcgtttcatctttagaagcgaatttcgccaatattataattatcaaaagagaggggtggcaaacggtatttggcattattaggttaaaaaatgtagaaggagagtgaaaccc

SEQ ID NO: 2 - DNA, 99 bp, Listeria monocytogenes
agaagcgaatttcgccaatattataattatcaaaagagaggggtggcaaacggtatttggcattattaggttaaaaaatgtagaaggagagtgaaaccc

SEQ ID NO: 3 - DNA, 65 bp, Listeria monocytogenes
agagaggggtggcaaacggtatttggcattattaggttaaaaaatgtagaaggagagtgaaaccc

SEQ ID NO: 4 - DNA, 20 bp, Listeria monocytogenes
gtagaaggagagtgaaaccc

SEQ ID NO: 5 - DNA, 150 bp, Listeria monocytogenes
taattcatgaatattttttcttatattagctaattaagaagataattaactgctaatccaatttttaacggaataaattagtgaaaatgaaggccgaattttccttgttctaaaaaggttgtattagcgtatcacgaggagggagtataa

SEQ ID NO: 6 - DNA, 99 bp, Listeria monocytogenes
gctaatccaatttttaacggaataaattagtgaaaatgaaggccgaattttccttgttctaaaaaggttgtattagcgtatcacgaggagggagtataa

SEQ ID NO: 7 - DNA, 70 bp, Listeria monocytogenes
gtgaaaatgaaggccgaattttccttgttctaaaaaggttgtattagcgtatcacgaggagggagtataa

SEQ ID NO: 8 - DNA, 20 bp, Listeria monocytogenes
atcacgaggagggagtataa

SEQ ID NO: 9 - DNA, 14 bp, Listeria monocytogenes
ttaacaattgttaa

SEQ ID NO: 10 - protein, 10 aa, Homo sapiens
RVAALARDAP

(19) United States
(12) Patent Application Publication
(10) Pub. No.: US 2005/0147621 A1
(43) Pub. Date: Jul. 7, 2005

(54) Use of bacterial 5' untranslated regions for nucleic acid expression

(76) Inventors: Darren E. Higgins, Jamaica Plain, MA (US); Aimee Shen, Boston, MA (US)

Correspondence Address: FISH & RICHARDSON PC, 225 FRANKLIN ST, BOSTON, MA, 02110, US

(21) Appl. No.: 10/961,291

(22) Filed: Oct. 8, 2004

Related U.S. Application Data
(60) Provisional application No. 60/510,599, filed on Oct. 10, 2003.

(51) Int. Cl. A61K 39/02; A61K 48/00; C07H 21/04; C12N 1/21

(52) U.S. Cl. 424/200.1; 514/44; 536/23.7; 435/252.3; 435/69.3

(57) ABSTRACT

Nucleic acids comprising bacterial untranslated regions and methods of using the nucleic acids are provided herein.

### CROSS-REFERENCE TO RELATED APPLICATIONS

This application claims the benefit of priority of U.S. Ser. No. 60/510,599, filed Oct. 10, 2003, the contents of which are hereby incorporated by reference in their entirety.

### TECHNICAL FIELD

This invention relates to compositions for nucleic acid expression, and more particularly to compositions comprising the 5′ untranslated regions of genes of Listeria monocytogenes and portions thereof and methods for their use in nucleic acid expression.

### BACKGROUND

Bacteria have many properties that are ideal for expression of recombinant proteins. Bacteria can be easy and inexpensive to culture. Culture systems can also be scaled up to produce large quantities of proteins. Methods for genetic manipulation are well established and numerous regulatable promoter systems are available. Escherichia coli is often used as a host strain for expression of recombinant proteins (Rudolph, R., 1996, Protein Engineering: Principles and Practice, Cleland, J. L. and Craik, C. S., eds., pp. 283-298, Wiley-Liss, New York). However, alternative bacterial strains may be useful when expression in Escherichia coli or a particular host strain is inefficient. For example, a recombinant protein may be toxic in one host, necessitating expression in a host that is not sensitive to the protein. Some bacterial hosts, such as E. coli, are unable to express high levels of particular proteins, or produce proteins in a partially folded, aggregated state (e.g., in inclusion bodies).

As an alternative to E. coli, Gram-positive bacterial strains can be used for production of recombinant proteins. For example, host strains of the genus Bacillus have been used for the production of a variety of proteins (Priest, Bacteriol. Rev., 41: 711 (1977); Glenn, Ann. Rev. Microbiol., 30: 41 (1976)). One Gram-positive host-vector expression system is based on the B. amyloliquefaciens alpha-amylase gene (Ulmanen et al., J. Bacteriol., 162: 176 (1985); Palva et al., Proc. Natl. Acad. Sci. U.S.A., 79: 5582 (1982); Palva et al., Gene, 22: 229 (1983); Lundstrom et al., Virus Res., 2: 69 (1985)). Expression systems based on other Gram-positive hosts such as B. subtilis, B. coagulans, B. licheniformis (U.S. Pat. Nos. 4,824,782, 5,171,673, and 4,711,843; WO 86/05812; Chang, S., Meth. Enzymol., 153: 507-516 (1987)), Lactococcus and Lactobacillus species (spp.). (U.S. Pat. No. 5,242,821), Staphylococcus spp. (Abrahmsen et al., EMBO J. 4: 3901-3906 (1986)), Streptomyces spp. (U.S. Pat. No. 4,745,056; JP Patent 2,002,379; EP 352,707) and Corynebacterium spp. (U.S. Pat. No. 4,965,197; WO 93/03158) have also been developed for general use.

### SUMMARY

The invention is based, in part, on the observation that fusion of all or a part of the Listeria monocytogenes actA or hly 5′ untranslated region (UTR) is sufficient to enhance expression of heterologous nucleic acids. The actA and hly 5′ UTRs can be used to enhance expression of heterologous nucleic acids, for example, in bacteria.

Accordingly, in one aspect, the invention features an isolated nucleic acid which includes: a 5′ UTR, wherein the 5′ UTR comprises a Listeria monocytogenes 5′ untranslated region (UTR), e.g., a Listeria monocytogenes 5′ UTR selected from the group consisting of a Listeria monocytogenes hly 5′ UTR, and a Listeria monocytogenes actA 5′ UTR, and functional fragments and variants thereof; optionally, a ribosome binding site (RBS); and a heterologous nucleic acid sequence, wherein the 5′ UTR is operably linked to the heterologous nucleic acid sequence. “Heterologous nucleic acid sequence”, as used herein, refers to a nucleic acid sequence that is not naturally associated with the Listeria monocytogenes 5′ UTR to which it is linked. In some embodiments, a heterologous nucleic acid sequence encodes a polypeptide. In one embodiment, the nucleic acid further includes a promoter. In one embodiment, the promoter is a bacterial promoter, e.g., a Gram-positive bacterial promoter. In one embodiment, the promoter is a bacteriophage promoter (e.g., a Listeria monocytogenes or Bacillus subtilis bacteriophage promoter). In another embodiment, the nucleic acid further includes a transcriptional activation site 5′ of the promoter, e.g., the transcriptional activation site is a prfA box. A prfA box is a DNA binding site found approximately 40 nucleotides 5′ (i.e., upstream) of prfA-regulated promoters. PrfA boxes are typically palindromic sequences of 14 nucleotides. An exemplary prfA box contains the following sequence: 5′ TTAACAATTGTTAA 3′ (See, e.g., Mengaud J, et al. Infect Immun. 57(12): 3695-701, 1989). Binding of a prfA box by the prfA gene product results in transcriptional activation of the gene associated with the prfA box (Freitag N E, et al. Infect Immun. 61(6): 2537-44, 1993).

In some embodiments, the RBS is a bacterial RBS, e.g., a Listeria monocytogenes RBS, e.g., the RBS that is naturally associated with the Listeria monocytogenes UTR. In other embodiments, the RBS is a heterologous RBS.

In one embodiment, the isolated nucleic acid includes more than one Listeria monocytogenes hly and/or actA UTR or functional fragments or variants thereof, e.g., more than one Listeria monocytogenes hly and/or actA UTR or functional fragments or variants thereof where the UTRs are operably linked.

In one embodiment, the Listeria monocytogenes 5′ UTR is the hly 5′ UTR or a functional fragment or variant thereof. In one embodiment, the 5′ UTR includes at least 65 contiguous nucleotides of the UTR immediately 5′ of the hly start codon. In another embodiment, the 5′ UTR includes a nucleotide sequence having at least 60%, 70%, 80%, 90%, 95% or more homology with the following sequence: AGAGAGGGGTGGCAAACGGTATTTGGCATTATTAGGTTAAAAAATGTAGAAGGAGAGTGAAACCC (SEQ ID NO:3). In another embodiment, the 5′ UTR differs from the sequence set forth in SEQ ID NO:3 by at least 1, 2, 3, 4 or 5 nucleotides but not more than 25 nucleotides. In some embodiments, the 5′ UTR includes the sequence set forth in SEQ ID NO:3.

In one embodiment, the Listeria monocytogenes 5′ UTR is the hly 5′ UTR or a functional fragment or variant thereof and the 5′ UTR includes at least 99 contiguous nucleotides of the UTR immediately 5′ of the hly start codon. In one embodiment, the 5′ UTR comprises a nucleotide sequence having at least 60%, 70%, 80%, 90%, 95% or more homology with the following sequence: AGAAGCGAATTTCGCCAATATTATAATTATCAAAAGAGAGGGGTGGCAAACGGTATTTGGCATTATTAGGTTAAAAAA TGTAGAAGGAGAGTGAAACCC (SEQ ID NO:2). In another embodiment, the 5′ UTR differs from the sequence set forth in SEQ ID NO:2 by at least 1, 2, 3, 5 or 10 nucleotides but not more than 25, 35, or 45 nucleotides. In some embodiments, the 5′ UTR comprises the nucleotide sequence set forth in SEQ ID NO:2.

In one embodiment, the Listeria monocytogenes 5′ UTR is the hly 5′ UTR or a functional fragment or variant thereof and the 5′ UTR includes 133 contiguous nucleotides of the UTR immediately 5′ of the hly start codon. In one embodiment, the hly 5′ UTR of the nucleic acid includes a nucleotide sequence having at least 60%, 70%, 80%, 90%, 95% or more homology with the following sequence: ATAAAGCAAGCATATAATATTGCGTTTCATCTTTAGAAGCGAATTTCGCCAATATTATAATTATCAAAAGAGAGGGGT GGCAAACGGTATTTGGCATTATTAGGTTAAAAAATGTAGAAGGAGAGTGAAACCC (SEQ ID NO:1). In one embodiment, the hly 5′ UTR differs from (SEQ ID NO:1) by at least 1, 2, 3, 5, but not more than 35 nucleotides. In one embodiment, nucleotides 1-113 of the sequence are at least 70%, 80%, 90%, 95% or more homologous to nucleotides 1-113 of SEQ ID NO:1. In one embodiment, the hly 5′ UTR includes the sequence set forth in SEQ ID NO:1.

In one embodiment, the Listeria monocytogenes 5′ UTR is the actA 5′ UTR or a functional fragment or variant thereof. In one embodiment, the 5′ UTR includes at least 70 contiguous nucleotides of the UTR immediately 5′ of the actA start codon. In one embodiment, the 5′ UTR includes a nucleotide sequence having at least 60%, 70%, 80%, 90%, 95% or more homology with the following sequence: GTGAAAATGAAGGCCGAATTTTCCTTGTTCTAAAAAGGTTGTATTAGCGTATCACGAGGAGGGAGTATAA (SEQ ID NO: 7). In one embodiment, the 5′ UTR includes the nucleotide sequence set forth in SEQ ID NO:7. In one embodiment, the 5′ UTR differs from SEQ ID NO:7 by at least 1, 2, 3, 4 or 5 nucleotides but not more than 25 or 35 nucleotides.

In another embodiment, the Listeria monocytogenes 5′ UTR is the actA 5′ UTR or a functional fragment or variant thereof and the 5′ UTR includes at least 99 contiguous nucleotides of the UTR immediately 5′ of the actA start codon. In one embodiment, the 5′ UTR includes a nucleotide sequence having at least 60%, 70%, 80%, 90%, 95% or more homology with the following sequence: GCTAATCCAATTTTTAACGGAATAAATTAGTGAAAATGAAGGCCGAATTTTCCTTGTTCTAAAAAGGTTGTATTAGC GTATCACGAGGAGGGAGTATAA (SEQ ID NO:6). In one embodiment, the 5′ UTR includes the sequence set forth in SEQ ID NO:6. In one embodiment, the 5′ UTR differs from SEQ ID NO:6: by at least 1, 2, 3, 4, 5 or 10 nucleotides but not more than 25, 35 or 45 nucleotides.

In one embodiment, the Listeria monocytogenes 5′ UTR is the actA 5′ UTR or a functional fragment or variant thereof and the 5′ UTR includes 150 contiguous nucleotides of the UTR immediately 5′ of the actA start codon. In one embodiment, the actA 5′ UTR includes a nucleotide sequence having at least 60%, 70%, 80%, 90%, 95% or more homology with the following sequence: TAATTCATGAATATTTTTTCTTATATTAGCTAATTAAGAAGATAATTAACTGCTAATCCAATTTTTAACGGAATAAA TTAGTGAAAATGAAGGCCGAATTTTCCTTGTTCTAAAAAGGTTGTATTAGCGTATCACGAGGAGGGAGTATAA (SEQ ID NO:5). In one embodiment, nucleotides 1-115 of the sequence are at least 70%, 80%, 90%, 95% or more homologous to nucleotides 1-115 of SEQ ID NO:5. In one embodiment, the 5′ UTR differs from SEQ ID NO:5 by at least 1, 2, 3, 4, 5 or 10 nucleotides but not more than 25, 35, 45 or 55 nucleotides. In one embodiment, the actA 5′ UTR comprises the sequence set forth in SEQ ID NO:5.

In one embodiment, the nucleic acid further includes at least one, preferably two, homologous recombination sequences which are homologous to genomic sequences of a host strain. In one embodiment, the homologous recombination sequence is at least 500, 1000, 2000, or 3000 nucleotides in length. In one embodiment, the nucleic acid includes two homologous recombination sequences and the homologous recombination sequences are of approximately equal length.

In one embodiment, the nucleic acid includes an integration site. In one embodiment, the integration site is a bacteriophage integrase site, e.g., a listeriophage integrase site.

In various embodiments, the heterologous nucleic acid encodes a nucleic acid (e.g., RNA) product. For example, the heterologous nucleic acid can encode an inhibitory RNA (RNAi) or fragment thereof (e.g., a single strand of a double-stranded RNAi). The heterologous nucleic acid can also specify other types of nucleic acid products, such as ribozymes.

In various embodiments, the heterologous nucleic acid sequence encodes a viral polypeptide or an antigenic fragment thereof. In one embodiment, the viral polypeptide is a viral polypeptide encoded by one of the following viruses: human immunodeficiency virus, hepatitis B virus, hepatitis C virus, hepatitis A virus, smallpox, influenza viruses, human papilloma viruses, adenoviruses, rhinoviruses, coronaviruses, herpes simplex virus, respiratory syncytial viruses, rabies, and coxsackie virus. In one embodiment, the viral polypeptide is encoded by the severe acute respiratory syndrome-associated coronavirus (SARS-CoV). In one embodiment, the viral polypeptide is chosen from the group consisting of the following: influenza antigens such as haemagglutinin (HA), nucleoprotein (NP), matrix protein (MP1); HIV antigens such as HIV gag, pol, env, tat, reverse transcriptase hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, and other Japanese encephalitis viral antigen components; rabies viral antigens such as rabies glycoprotein, rabies nucleoprotein and other rabies viral antigen components; and Hepatitis B surface antigen.

In various embodiments, the heterologous nucleic acid sequence encodes a mammalian polypeptide. In one embodiment, the mammalian polypeptide is a cancer-associated polypeptide or an antigenic fragment thereof. In one embodiment, the cancer-associated polypeptide is chosen from the group consisting of: 707 alanine proline peptide (707-AP; the sequence of 707-AP is RVAALARDAP (Takahashi T, et al., Clin Cancer Res. 3(8): 1363-70, 1997)); alpha (α)-fetoprotein (AFP); adenocarcinoma antigen recognized by T cells 4 (ART-4); B antigen (BAGE); β-catenin/mutated(b-catenin/m); breakpoint cluster region-Abelson (Bcr-abl); CTL-recognized antigen on melanoma (CAMEL); carcinoembryonic antigen peptide-1 (CAP-1); caspase-8 (CASP-8); cell-division cycle 27 mutated (CDC27m); cycline-dependent kinase 4 mutated CDK4/m); carcinoembryonic antigen (CEA); cancer/testis (CT) antigen; cyclophilin B (Cyp-B); differentiation antigen melanoma (DAM-6, also known as MAGE-B2, and DAM-10, also known as MAGE-B1); elongation factor 2 mutated (ELF2M); Ets variant gene 6/acute myeloid leukemia 1 gene ETS (ETV6-AML1); glycoprotein 250 (G250); G antigen (GAGE); N-acetylglucosaminyltransferase V (GnT-V); glycoprotein 100 kD (GnT-V); helicase antigen (HAGE); human epidermal receptor-2/neurological (HER-2/neu); HLA-A*0201-R1701 (HLA-A*0201 having an arginine (R) to isoleucine (I) exchange at residue 170 of the α-helix of the α2-domain in the HLA-A2 gene); human papilloma virus E7 (HPV-E7); human papilloma virus E6 (HPV-E6); heat shock protein 70-2 mutated (HSP70-2M); human signet ring tumor-2 (HST-2); human telomerase reverse transcriptase (hTERT or hTRT); intestinal carboxyl esterase (iCE); KIAA0205; L antigen (LAGE); low density lipid receptor/GDP-L-fucose: β-D-galactosidase 2-α-L-fucosyltransferase (LDLR/FUT); melanoma antigen (MAGE); melanoma antigen recognized by T cells-1/Melanoma antigen A (MART-1/Melan-A); melanocortin 1 receptor (MC1R); myosin mutated (Myosin/m); mucin 1 (MUC 1); melanoma ubiquitous mutated 1 (MUM-1), melanoma ubiquitous mutated 2 (MUM-2), melanoma ubiquitous mutated 3 (MUM-3); New York-esophageous 1 (NY-ESO-1); protein 15 (P15); protein of 190 KD bcr-abl (p190 minor bcr-abl); promyelocytic leukaemia/retinoic acid receptor α (Pml/RARa); preferentially expressed antigen of melanoma (PRAME); prostate-specific antigen (PSA); prostate-specific membrane antigen (PSM); renal antigen (RAGE); renal ubiquitous 1 (RU1), renal ubiquitous 2 (RU2); sarcoma antigen (SAGE); squamous antigen recognized by T cells-1 (SART-1); squamous antigen recognized by T cells-3 (SART-3); translocation Ets-family leukemia/acute myeloid leukemia 1 (TEL/AML1); triosephosphate isomerase mutated (TPI/m); tyrosinase related protein 1 (TRP-1 or gp75); tyrosinase related protein 2 (TRP2); TRP-2/intron 2 (TRP-2/INT2); Wilms' tumor gene (WT-1).

In various embodiments, the heterologous nucleic acid sequence encodes a bacterial polypeptide or an antigenic fragment thereof. In one embodiment, the bacterial polypeptide is a bacterial polypeptide encoded by one of the following bacteria: Mycobacterium spp. (e.g., Mycobacterium tuberculosis, Mycobacterium leprae), Streptococcus spp. (e.g., Streptococcus pneumoniae, Streptococcus pyogenes), Staphylococcus spp. (e.g., Staphylococcus aureus), Treponema (e.g., Treponema pallidum), Chlamydia spp., Vibrio spp. (e.g., Vibrio cholerae), Bacillus spp. (e.g., Bacillus subtilis, Bacillus anthracis), Yersinia spp. (e.g., Yersinia pestis), Neisseria spp. (e.g., Neisseria meningitides, Neisseria gonorrhoeae), Legionella spp., Bordetella spp. (e.g., Bordetella pertussis), Shigella spp., Campylobacter spp., Pseudomonas spp. (e.g., Pseudomonas aeruginosa), Brucella spp., Clostridium spp. (e.g., Clostridium tetani, Clostridium botulinum, Clostridium perfringens), Salmonella spp. (e.g., Salmonella typhi), Borrelia spp. (e.g., Borrelia burgdorferi), Rickettsia spp. (e.g., Rickettsia prowazeki), Mycoplasma spp. (e.g., Mycoplasma pneumoniae), Haemophilus spp. (e.g., Haemophilus influenzae), Branhamella spp. (e.g., Branhamella catarrhalis), Corynebacteria spp. (e.g., Corynebacteria diphtheriae), Klebsiella spp. (e.g., Klebsiella pneumoniae), Escherichia spp. (e.g., Escherichia coli), and Listeria spp. (e.g., Listeria monocytogenes).

In one embodiment, the bacterial polypeptide is chosen from the group consisting of: listeriolysin O, L. monocytogenes p60, L. monocytogenes metalloprotease (MPL), Chlamydia Cap1, Chlamydia Cap2, M. tuberculosis heat shock protein (hsp)60, M. tuberculosis hsp70, M. tuberculosis Ag85, M. tuberculosis ESAT-6 and M. tuberculosis CFP10.

In various embodiments, the heterologous nucleic acid sequence encodes a parasitic or fungal polypeptide. In one embodiment, the parasitic or fungal polypeptide is a polypeptide encoded by one of the following parasites or fungi: Candida spp. (e.g., Candida albicans), Cryptococcus spp. (e.g., Cryptococcus neoformans), Aspergillus spp., Histoplasma spp. (e.g., Histoplasma capsulatum), Coccidioides spp. (e.g., Coccidioides immitis), Pneumocystis (e.g., Pneumocystis carinii), Entamoeba spp. (e.g., Entamoeba histolytica), Giardia spp., Leishmania spp., Plasmodium spp., Trypanosoma spp., Toxoplasma spp. (e.g., Toxoplasma gondii), Cryptosporidium spp., Trichuris spp. (e.g., Trichuris trichiura), Trichinella spp. (e.g., Trichinella spiralis), Enterobius spp. (e.g., Enterobius vermicularis), Ascaris spp. (e.g., Ascaris lumbricoides), Ancylostoma spp., Stongyloides spp., Filaria spp., and Schistosoma spp. In one embodiment, the parasitic polypeptide is chosen from the group consisting of: MSP-1; malarial antigens 41-3, AMA-1, CSP, PFEMP-1, GBP-130, MSP-1, PFS-16, SERP; fungal antigens such as heat shock protein 60; plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

In one embodiment, the Listeria monocytogenes 5′ UTR increases expression of a polypeptide encoded by the heterologous nucleic acid sequence relative to the expression of the polypeptide encoded by the heterologous nucleic acid sequence when the sequence encoding the polypeptide is not operably linked to the UTR. In one embodiment, the Listeria monocytogenes 5′ UTR increases expression of the polypeptide at least 1.5-fold, 2-fold, 5-fold, 10-fold, 30-fold, or 50-fold relative to the expression of the polypeptide when the sequence encoding the polypeptide is not operably linked to the UTR.

In another aspect, the invention features an isolated nucleic acid consisting of a Listeria monocytogenes 5′ untranslated region (UTR) selected from the group consisting of a Listeria monocytogenes hly 5′ UTR, a Listeria monocytogenes actA 5′ UTR, both a Listeria monocytogenes hly 5′ UTR and a Listeria monocytogenes actA 5′ UTR, and functional fragments and variants thereof.

In one embodiment, the 5′ UTR includes a ribosome binding site (RBS). In one embodiment, the RBS is a bacterial RBS. In one embodiment, the RBS is the RBS that is naturally associated with the UTR. In one embodiment, the RBS is a heterologous RBS.

In one embodiment, the Listeria monocytogenes 5′ UTR is the hly 5′ UTR or a functional fragment or variant thereof. In one embodiment, the 5′ UTR includes at least 65 contiguous nucleotides of the UTR immediately 5′ of the hly start codon. In one embodiment, the 5′ UTR includes a nucleotide sequence having at least 60%, 70%, 80%, 90%, 95% or more homology with the following sequence: AGAGAGGGGTGGCAAACGGTATTTGGCATTATTAGGTTAAAAAATGTAGAAGGAGAGTGAAACCC (SEQ ID NO:3). In one embodiment, the 5′ UTR includes the sequence set forth in SEQ ID NO:3. In one embodiment, the 5′ UTR includes a nucleic acid sequence which differs by at least 1, 2, 3, 4 or 5 but not more than 25 or 35 nucleotides from SEQ ID NO:3.

In one embodiment, the Listeria monocytogenes 5′ UTR is the hly 5′ UTR or a functional fragment or variant thereof and the 5′ UTR includes at least 99 contiguous nucleotides of the UTR immediately 5′ of the hly start codon. In one embodiment, the 5′ UTR includes a nucleotide sequence having at least 60%, 70%, 80%, 90%, 95% or more homology with the following sequence: AGAAGCGAATTTCGCCAATATTATAATTATCAAAAGAGAGGGGTGGCAAACGGTATTTGGCATTATTAGGTTAAAAAA TGTAGAAGGAGAGTGAAACCC (SEQ ID NO:2). In one embodiment, the 5′ UTR includes the nucleotides sequence set forth in SEQ ID NO:2. In one embodiment, the 5′ UTR includes a nucleic acid sequence which differs by at least 1, 2, 3, 4, 5 or 10 but not more than 25, 35 or 45 nucleotides from SEQ ID NO:2.

In one embodiment, the Listeria monocytogenes 5′ UTR is the hly 5′ UTR or a functional fragment or variant thereof and the 5′ UTR includes 133 contiguous nucleotides of the UTR immediately 5′ of the hly start codon. In one embodiment, the hly 5′ UTR includes a nucleotide sequence having at least 60%, 70%, 80%, 90%, 95% or more homology to the following sequence: ATAAAGCAAGCATATAATATTGCGTTTCATCTTTAGAAGCGAATTTCGCCAATATTATAATTATCAAAAGAGAGGGGT GGCAAACGGTATTTGGCATTATTAGGTTAAAAAATGTAGAAGGAGAGTGAAACCC (SEQ ID NO:1). In one embodiment, the hly 5′ UTR consists of a nucleotide sequence that differs from (SEQ ID NO:1) by at least 1, 2, 3, 5 or 10 but not more than 25, 35, 45 or 55 nucleotides. In one embodiment, nucleotides 1-90 of the sequence are at least 70%, 80%, 90%, 95% or more homologous to nucleotides 1-113 of SEQ ID NO:1. In one embodiment, the hly 5′ UTR consists of the sequence set forth in SEQ ID NO:1.

In one embodiment, the Listeria monocytogenes 5′ UTR is the actA 5′ UTR or a functional fragment or variant thereof. In one embodiment, the 5′ UTR includes at least 70 contiguous nucleotides of the UTR immediately 5′ of the actA start codon. In one embodiment, the 5′ UTR includes a nucleotide sequence having at least 60%, 70%, 80%, 90%, 95% or more homology to the following sequence: GTGAAAATGAAGGCCGAATTTTCCTTGTTCTAAAAAGGTTGTATTAGCGTATCACGAGGAGGGAGTATAA (SEQ ID NO:7). In one embodiment, the 5′ UTR includes the nucleotide sequence set forth in SEQ ID NO:7. In one embodiment, the 5′ UTR includes a nucleic acid sequence which differs by at least 1, 2, 3, 4, or 5 but not more than 25 or 35 nucleotides from SEQ ID NO:7.

In one embodiment, the Listeria monocytogenes 5′ UTR is the actA 5′ UTR or a functional fragment or variant thereof and the 5′ UTR includes at least 99 contiguous nucleotides of the UTR immediately 5′ of the actA start codon. In one embodiment, the 5′ UTR includes a nucleotide sequence having at least 60%, 70%, 80%, 90%, 95% or more homology to the following sequence: GCTAATCCAATTTTTAACGGAATAAATTAGTGAAAATGAAGGCCGAATTTTCCTTGTTCTAAAAAGGTTGTATTAGC GTATCACGAGGAGGGAGTATAA (SEQ ID NO:6). In one embodiment, the 5′ UTR includes the sequence set forth in SEQ ID NO:6. In one embodiment, the 5′ UTR includes a nucleic acid sequence which differs by at least 1, 2, 3, 4, 5 or 10 but not more than 25, 35 or 45 nucleotides from SEQ ID NO:6:.

In one embodiment, the Listeria monocytogenes 5′ UTR is the actA 5′ UTR or a functional fragment or variant thereof and the 5′ UTR includes 150 contiguous nucleotides of the UTR immediately 5′ of the actA start codon. In one embodiment, the actA 5′ UTR includes a nucleotide sequence having at least 60%, 70%, 80%, 90%, 95% or more homology to the following sequence: TAATTCATGAATATTTTTTCTTATATTAGCTAATTAAGAAGATAATTAACTGCTAATCCAATTTTTAACGGAATAAA TTAGTGAAAATGAAGGCCGAATTTTCCTTGTTCTAAAAAGGTTGTATTAGCGTATCACGAGGAGGGAGTATAA (SEQ ID NO:5). In one embodiment, nucleotides 1-115 of the sequence are at least 70%, 80%, 90%, 95% or more homologous to nucleotides 1-111 of SEQ ID NO:5. In one embodiment, the actA 5′ UTR consists of the sequence set forth in SEQ ID NO:5. In one embodiment, the 5′ UTR consists of a nucleic acid sequence which differs by at least 1, 2, 3, 4, 5 or 10 but not more than 25, 35, 45 or 55 nucleotides from SEQ ID NO:5.

In one embodiment, the nucleic acid further includes one or more restriction endonuclease sites 5′ and/or 3′ of the UTR.

In another aspect, the invention features a nucleic acid vector which includes a nucleic acid described herein.

In another aspect, the invention features a nucleic acid vector that includes: a promoter, e.g., a Listeria monocytogenes promoter; a Listeria monocytogenes hly 5′ untranslated region (UTR), wherein the UTR, optionally, includes a ribosome binding site. The vector can further include one or more of: a heterologous nucleic acid sequence; an integration site; a selectable marker; and a bacterial origin of replication. When the vector includes a promoter and a heterologous nucleic acid sequence, the UTR is operably linked to the promoter and the heterologous nucleic acid sequence.

In another aspect, the invention features a nucleic acid vector that includes: a promoter, e.g., a Listeria monocytogenes promoter; a Listeria monocytogenes actA 5′ untranslated region (UTR), wherein the UTR optionally includes a ribosome binding site. The vector can further include one or more of: a heterologous nucleic acid sequence; an integration site; a selectable marker; and a bacterial origin of replication. When the vector includes a promoter and a heterologous nucleic acid sequence, the UTR is operably linked to the promoter and the sequence encoding the polypeptide.

In another aspect, the invention features a bacterium which includes: a nucleic acid which includes a promoter; a 5′ UTR, wherein the 5′ UTR includes a Listeria monocytogenes 5′ UTR, and a ribosome binding site; and a heterologous nucleic acid sequence; wherein the UTR is operably linked to the promoter and the heterologous nucleic acid sequence, and wherein the Listeria monocytogenes 5′ untranslated region (UTR) is selected from the group consisting of a Listeria monocytogenes hly 5′ UTR, a Listeria monocytogenes actA 5′ UTR, and functional fragments and variants thereof.

In one embodiment, the bacterium is a Gram-positive bacterium. In one embodiment, the bacterium is selected from the group consisting of: a Listeria monocytogenes bacterium, a Bacillus subtilis bacterium, and a Lactococcus lactis bacterium. In another embodiment, the bacterium is a Gram-negative bacterium. In one embodiment, the isolated nucleic acid includes more than one Listeria monocytogenes hly and/or actA UTR or functional fragments or variants thereof, e.g., more than one Listeria monocytogenes hly and/or actA UTR and/or functional fragments and/or variants thereof where the UTRs are operably linked.

In one embodiment, the Listeria monocytogenes 5′ UTR is the hly 5′ UTR or a functional fragment or variant thereof. In one embodiment, the 5′ UTR includes at least 65 contiguous nucleotides of the UTR immediately 5′ of the hly start codon. In another embodiment, the 5′ UTR includes a nucleotide sequence having at least 60%, 70%, 80%, 90%, 95% or more homology with the following sequence: AGAGAGGGGTGGCAAACGGTATTTGGCATTATTAGGTTAAAAAATGTAGAAGGAGAGTGAAACCc (SEQ ID NO:3). In another embodiment, the 5′ UTR differs from the sequence set forth in SEQ ID NO:3 by at least 1, 2, 3, 4 or 5 nucleotides but not more than 25 nucleotides. In some embodiments, the 5′ UTR includes the sequence set forth in SEQ ID NO:3.

In one embodiment, the Listeria monocytogenes 5′ UTR is the hly 5′ UTR or a functional fragment or variant thereof and the 5′ UTR includes at least 99 contiguous nucleotides of the UTR immediately 5′ of the hly start codon. In one embodiment, the 5′ UTR comprises a nucleotide sequence having at least 60%, 70%, 80%, 90%, 95 or more homology with the following sequence: AGAAGCGAATTTCGCCAATATTATAATTATCAAAAGAGAGGGGTGGCAAACGGTATTTGGCATTATTAGGTTAAAAAA TGTAGAAGGAGAGTGAAACCC (SEQ ID NO:2). In another embodiment, the 5′ UTR differs from the sequence set forth in SEQ ID NO:2 by at least 1, 2, 3, 5 or 10 nucleotides but not more than 25, 35, or 45 nucleotides. In some embodiments, the 5′ UTR comprises the nucleotide sequence set forth in SEQ ID NO:2.

In one embodiment, the Listeria monocytogenes 5′ UTR is the hly 5′ UTR or a functional fragment or variant thereof and the 5′ UTR includes 133 contiguous nucleotides of the UTR immediately 5′ of the hly start codon. In one embodiment, the hly 5′ UTR of the nucleic acid includes a nucleotide sequence having at least 60%, 70%, 80%, 90%, 95% or more homology with the following sequence: ATAAAGCAAGCATATAATATTGCGTTTCATCTTTAGAAGCGAATTTCGCCAATATTATAATTATCAAAAGAGAGGGGT GGCAAACGGTATTTGGCATTATTAGGTTAAAAAATGTAGAAGGAGAGTGAAACCC (SEQ ID NO:1). In one embodiment, the hly 5′ UTR differs from (SEQ ID NO:1) by at least 1, 2, 3, 5, but not more than 35 nucleotides. In one embodiment, nucleotides 1-113 of the sequence are at least 70%, 80%, 90%, 95% or more homologous to nucleotides 1-113 of SEQ ID NO:1. In one embodiment, the hly 5′ UTR includes the sequence set forth in SEQ ID NO:1.

In one embodiment, the Listeria monocytogenes 5′ UTR is the actA 5′ UTR or a functional fragment or variant thereof. In one embodiment, the 5′ UTR includes at least 70 contiguous nucleotides of the UTR immediately 5′ of the actA start codon. In one embodiment, the 5′ UTR includes a nucleotide sequence having at least 60%, 70%, 80%, 90%, 95% or more homology with the following sequence: GTGAAAATGAAGGCCGAATTTTCCTTGTTCTAAAAAGGTTGTATTAGCGTATCACGAGGAGGGAGTATAA (SEQ ID NO:7). In one embodiment, the 5′ UTR includes the nucleotide sequence set forth in SEQ ID NO:7. In one embodiment, the 5′ UTR differs from SEQ ID NO:7 by at least 1, 2, 3, 4 or 5 nucleotides but not more than 25 or 35 nucleotides.

In another embodiment, the Listeria monocytogenes 5′ UTR is the actA 5′ UTR or a functional fragment or variant thereof and the 5′ UTR includes at least 99 contiguous nucleotides of the UTR immediately 5′ of the actA start codon. In one embodiment, the 5′ UTR includes a nucleotide sequence having at least 60%, 70%, 80%, 90%, 95% or more homology with the following sequence: GCTAATCCAATTTTTAACGGAATAAATTAGTGAAAATGAAGGCCGAATTTTCCTTGTTCTAAAAAGGTTGTATTAGC GTATCACGAGGAGGGAGTATAA (SEQ ID NO:6). In one embodiment, the 5′ UTR includes the sequence set forth in SEQ ID NO:6. In one embodiment, the 5′ UTR differs from SEQ ID NO:6 by at least 1, 2, 3, 4, 5 or 10 nucleotides but not more than 25, 35 or 45 nucleotides.

In one embodiment, the Listeria monocytogenes 5′ UTR is the actA 5′ UTR or a functional fragment or variant thereof and the 5′ UTR includes 150 contiguous nucleotides of the UTR immediately 5′ of the actA start codon. In one embodiment, the actA 5′ UTR includes a nucleotide sequence having at least 60%, 70%, 80%, 90%, 95% or more homology with the following sequence: TAATTCATGAATATTTTTTCTTATATTAGCTAATTAAGAAGATAATTAACTGCTAATCCAATTTTTAACGGAATAAA TTAGTGAAAATGAAGGCCGAATTTTCCTTGTTCTAAAAAGGTTGTATTAGCGTATCACGAGGAGGGAGTATAA (SEQ ID NO:5). In one embodiment, nucleotides 1-115 of the sequence are at least 70%, 80%, 90%, 95% or more homologous to nucleotides 1-115 of SEQ ID NO:5. In one embodiment, the 5′ UTR differs from SEQ ID NO:5 by at least 1, 2, 3, 4, 5 or 10 nucleotides but not more than 25, 35, 45 or 55 nucleotides. In one embodiment, the actA 5′ UTR comprises the sequence set forth in SEQ ID NO:5.

In one embodiment, the promoter is a bacterial promoter. In one embodiment, the promoter is a Gram-positive bacterial promoter. In one embodiment, the promoter is a Gram-negative bacterial promoter. In one embodiment, the promoter is a bacteriophage promoter (e.g., a Listeria monocytogenes or Bacillus subtilis bacteriophage promoter).

In one embodiment, the nucleic acid further includes at least one, preferably two, homologous recombination sequences which are homologous to genomic sequences of a host strain (e.g., a homologous recombination sequence is 5′ of the promoter sequence and a homologous recombination sequence which is 3′ of the sequence encoding the polypeptide). In one embodiment, the homologous recombination sequence is at least 500, 1000, 2000, or 3000 nucleotides in length. In one embodiment, the nucleic acid includes two homologous recombination sequences and the homologous recombination sequences are of approximately equal length.

In one embodiment, the nucleic acid includes an integration site. In one embodiment, the integration site is a bacteriophage integrase site, e.g., a listeriophage integrase site.

In various embodiments, the heterologous nucleic acid sequence encodes a viral polypeptide or an antigenic fragment thereof. In one embodiment, the viral polypeptide is a viral polypeptide encoded by one of the following viruses: human immunodeficiency virus, hepatitis B virus, hepatitis C virus, hepatitis A virus, smallpox, influenza viruses, human papilloma viruses, adenoviruses, rhinoviruses, coronaviruses, herpes simplex virus, respiratory syncytial viruses, rabies, and coxsackie virus. In one embodiment, the polypeptide is encoded by SARS-CoV. In one embodiment, the viral polypeptide is chosen from the group consisting of the following: influenza antigens such as haemagglutinin (HA), nucleoprotein (NP), matrix protein (MP1); HIV antigens such as HIV gag, pol, env, tat, reverse transcriptase hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, and other Japanese encephalitis viral antigen components; rabies viral antigens such as rabies glycoprotein, rabies nucleoprotein and other rabies viral antigen components; and Hepatitis B surface antigen.

In various embodiments, the heterologous nucleic acid sequence encodes a mammalian polypeptide. In one embodiment, the mammalian polypeptide is a cancer-associated polypeptide or an antigenic fragment thereof. In one embodiment, the cancer-associated polypeptide is chosen from the group consisting of: 707 alanine proline (707-AP); alpha (α)-fetoprotein (AFP); adenocarcinoma antigen recognized by T cells 4 (ART-4); B antigen (BAGE); β-catenin/mutated(b-catenin/m); breakpoint cluster region-Abelson (Bcr-abl); CTL-recognized antigen on melanoma (CAMEL); carcinoembryonic antigen peptide-1 (CAP-1); caspase-8 (CASP-8); cell-division cycle 27 mutated (CDC27m); cycline-dependent kinase 4 mutated CDK4/m); carcinoembryonic antigen (CEA); cancer/testis (CT) antigen; cyclophilin B (Cyp-B); differentiation antigen melanoma (DAM-6, also known as MAGE-B2, and DAM-10, also known as MAGE-B1); elongation factor 2 mutated (ELF2M); Ets variant gene 6/acute myeloid leukemia 1 gene ETS (ETV6-AML1); glycoprotein 250 (G250); G antigen (GAGE); N-acetylglucosaminyltransferase V (GnT-V); glycoprotein 100 kD (GnT-V); helicase antigen (HAGE); human epidermal receptor-2/neurological (HBER-2/neu); HLA-A*0201-R1701 (HLA-A*0201 having an arginine (R) to isoleucine (I) exchange at residue 170 of the α-helix of the α2-domain in the HLA-A2 gene); human papilloma virus E7 (HPV-E7); human papilloma virus E6 (HPV-E6); heat shock protein 70-2 mutated (HSP70-2M); human signet ring tumor-2 (HST-2); human telomerase reverse transcriptase (hTERT or hTRT); intestinal carboxyl esterase (iCE); KIAA0205; L antigen (LAGE); low density lipid receptor/GDP-L-fucose: β-D-galactosidase 2-α-Lfucosyltransferase (LDLR/FUT); melanoma antigen (MAGE); melanoma antigen recognized by T cells-1/Melanoma antigen A (MART-1/Melan-A); melanocortin 1 receptor (MC1R); myosin mutated (Myosin/m); mucin 1 (MUC 1); melanoma ubiquitous mutated 1 (MUM-1), melanoma ubiquitous mutated 2 (MUM-2), melanoma ubiquitous mutated 3 (MUM-3); New York-esophageous 1 (NY-ESO-1); protein 15 (P15); protein of 190 KD bcr-abl (p190 minor bcr-abl); promyelocytic leukaemia/retinoic acid receptor α (Pml/RARa); preferentially expressed antigen of melanoma (PRAME); prostate-specific antigen (PSA); prostate-specific membrane antigen (PSM); renal antigen (RAGE); renal ubiquitous 1 (RU1), renal ubiquitous 2 (RU2); sarcoma antigen (SAGE); SART-1; SART-3; translocation Ets-family leukemia/acute myeloid leukemia 1 (TEL/AML1); triosephosphate isomerase mutated (TPI/m); tyrosinase related protein 1 (TRP-1 or gp75); tyrosinase related protein 2 (TRP2); TRP-2/intron 2 (TRP-2/INT2); Wilms' tumor gene (WT-1).

In various embodiments, the heterologous nucleic acid sequence encodes a bacterial polypeptide or an antigenic fragment thereof. In one embodiment, the bacterial polypeptide is a bacterial polypeptide encoded by one of the following bacteria: Mycobacterium spp. (e.g., Mycobacterium tuberculosis, Mycobacterium leprae), Streptococcus spp. (e.g., Streptococcus pneumoniae, Streptococcus pyogenes), Staphylococcus spp. (e.g., Staphylococcus aureus), Treponema (e.g., Treponema pallidum), Chlamydia spp., Vibrio spp. (e.g., Vibrio cholerae), Bacillus spp. (e.g., Bacillus subtilis, Bacillus anthracis), Yersinia spp. (e.g., Yersinia pestis), Neisseria spp. (e.g., Neisseria meningitides, Neisseria gonorrhoeae), Legionella spp., Bordetella spp. (e.g., Bordetella pertussis), Shigella spp., Campylobacter spp., Pseudomonas spp. (e.g., Pseudomonas aeruginosa), Brucella spp., Clostridium spp. (e.g., Clostridium tetani, Clostridium botulinum, Clostridium perfringens), Salmonella spp. (e.g., Salmonella typhi), Borrelia spp. (e.g., Borrelia burgdorferi), Rickettsia spp. (e.g., Rickettsia prowazeki), Mycoplasma spp. (e.g., Mycoplasma pneumoniae), Haemophilus spp. (e.g., Haemophilus influenzae), Branhamella spp. (e.g., Branhamella catarrhalis), Corynebacteria spp. (e.g., Corynebacteria diphtheriae), Klebsiella spp. (e.g., Klebsiella pneumoniae), Escherichia spp. (e.g., Escherichia coli), and Listeria spp. (e.g., Listeria monocytogenes).

In one embodiment, the bacterial polypeptide is chosen from the group consisting of: listeriolysin 0, L. monocytogenes p60, L. monocytogenes metalloprotease (MPL), Chlamydia Cap1, Chlamydia Cap2, M. tuberculosis heat shock protein (hsp)60, M. tuberculosis hsp70, M. tuberculosis Ag85, M. tuberculosis ESAT-6 and M. tuberculosis CFP10.

In various embodiments, the heterologous nucleic acid sequence encodes a parasitic or fungal polypeptide. In one embodiment, the parasitic or fungal polypeptide is a polypeptide encoded by one of the following parasites or fungi: Candida spp. (e.g., Candida albicans), Cryptococcus spp. (e.g., Cryptococcus neoformans), Aspergillus spp., Histoplasma spp. (e.g., Histoplasma capsulatum), Coccidioides spp. (e.g., Coccidioides immitis), Pneumocystis (e.g., Pneumocystis carinii), Entamoeba spp. (e.g., Entamoeba histolytica), Giardia spp., Leishmania spp., Plasmodium spp., Trypanosoma spp., Toxoplasma spp. (e.g., Toxoplasma gondii), Cryptosporidium spp., Trichuris spp. (e.g., Trichuris trichiura), Trichinella spp. (e.g., Trichinella spiralis), Enterobius spp. (e.g., Enterobius vermicularis), Ascaris spp. (e.g., Ascaris lumbricoides), Ancylostoma spp., Stongyloides spp., Filaria spp., and Schistosoma spp. In one embodiment, the parasitic polypeptide is chosen from the group consisting of: MSP-1; malarial antigens 41-3, AMA-1, CSP, PFEMP-1, GBP-130, MSP-1, PFS-16, SERP; fungal antigens such as heat shock protein 60; plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

In one embodiment, the Listeria monocytogenes 5′ UTR increases expression of a polypeptide encoded by the heterologous nucleic acid sequence relative to a polypeptide encoded by the heterologous nucleic acid sequence that is not operably linked to the UTR. In one embodiment, the Listeria monocytogenes 5′ UTR increases expression of the polypeptide at least 1.5-fold, 2-fold, 5-fold, 10-fold, 30-fold, or 50-fold relative to a polypeptide that is not operably linked to the UTR.

In one embodiment, the bacterium expresses a cytolysin. In one embodiment, the cytolysin is listeriolysin O. In one embodiment, the cytolysin is non-secreted.

In one embodiment, the bacterium is nonvirulent and nonpathogenic.

In one embodiment, the bacterium includes a heterologous nucleic acid encoding a functional cytolysin such that the bacterium expresses a functional cytolysin. In one embodiment, the cytolysin is listeriolysin O.

In another aspect, the invention features a vaccine that includes a bacterium described herein.

In another embodiment, the invention features a vaccine comprising a nucleic acid described herein.

In another aspect, the invention features a pharmaceutical composition comprising a bacterium described herein.

In another aspect, the invention features a method for making a bacterium. The method includes, for example, the steps of: introducing into the bacterium a nucleic acid described herein.

In another aspect, the invention features a method for introducing an antigen into a eukaryotic cell. The method can include, for example: contacting the cells with a bacterium, wherein the bacterium includes a nucleic acid comprising: a promoter; a 5′ UTR, wherein the 5′ UTR includes a Listeria monocytogenes 5′ UTR and an RBS; and a heterologous nucleic acid sequence, wherein the UTR is operably linked to the RBS and the heterologous nucleic acid sequence. In one embodiment, the Listeria monocytogenes 5′ untranslated region (UTR) is selected from the group consisting of a Listeria monocytogenes hly 5′ UTR, a Listeria monocytogenes actA 5′ UTR, both a Listeria monocytogenes hly 5′ UTR and a Listeria monocytogenes actA 5′ UTR, and functional fragments and variants thereof.

In one embodiment, the bacterium expresses a cytolysin.

In one embodiment, the bacterium is nonpathogenic and nonvirulent.

In another aspect, the invention features a method for introducing an antigen into a eukaryotic cell. The method can include, for example: contacting the cells with a nucleic acid comprising: a promoter; a 5′ UTR, wherein the 5′ UTR includes a Listeria monocytogenes 5′ UTR and an RBS; and a heterologous nucleic acid sequence, wherein the UTR is operably linked to the RBS and the heterologous nucleic acid sequence. In one embodiment, the Listeria monocytogenes 5′ untranslated region (UTR) is selected from the group consisting of a Listeria monocytogenes hly 5′ UTR, a Listeria monocytogenes actA 5′ UTR, both a Listeria monocytogenes hly 5′ UTR and a Listeria monocytogenes actA 5′ UTR, and functional fragments and variants thereof.

In another aspect, the invention features a method for inducing an immune response to an antigen in a subject. The method includes, for example: administering to the subject a plurality of bacteria, wherein each bacterium includes a nucleic acid, comprising: a promoter; a 5′ UTR, wherein the 5′ UTR includes a Listeria monocytogenes 5′ UTR and an RBS; and a heterologous nucleic acid sequence, wherein the UTR is operably linked to the RBS and the heterologous nucleic acid sequence. In one embodiment, the Listeria monocytogenes 5′ untranslated region (UTR) is selected from the group consisting of a Listeria monocytogenes hly 5′ UTR, a Listeria monocytogenes actA 5′ UTR, both a Listeria monocytogenes hly 5′ UTR and a Listeria monocytogenes actA 5′ UTR, and functional fragments and variants thereof. In one embodiment, more than 20% (e.g., 20%, 30%, 40%, 50%, 60%, 70%, 80%, 90%, 95%, 97%, 98%, 99%,) of the bacteria of the plurality includes a polypeptide encoded by the heterologous nucleic acid sequence (i.e., the bacteria are “preloaded”, and, e.g., can be administered in inactivated/attenuated form).

In another aspect, the invention features a method for inducing an immune response to an antigen in a subject. The method includes, for example: administering to the subject a nucleic acid comprising: a promoter; a 5′ UTR, wherein the 5′ UTR includes a Listeria monocytogenes 5′ UTR and an RBS; and a heterologous nucleic acid sequence, wherein the UTR is operably linked to the RBS and the heterologous nucleic acid sequence. In one embodiment, the Listeria monocytogenes 5′ untranslated region (UTR) is selected from the group consisting of a Listeria monocytogenes hly 5′ UTR, a Listeria monocytogenes actA 5′ UTR, both a Listeria monocytogenes hly 5′ UTR and a Listeria monocytogenes actA 5′ UTR, and functional fragments and variants thereof.

In another aspect, the invention features a method for expressing a polypeptide. The method includes, for example: introducing into a bacterium a nucleic acid described herein, wherein the heterologous nucleic sequence encodes the polypeptide, and expressing the polypeptide.

Heterologous polypeptide or heterologous antigen, as used herein, refers to a protein or peptide encoded by a heterologous nucleic acid. As described above, a heterologous nucleic acid refers to a nucleic acid which is not normally linked to a specified 5′ UTR from Listeria monocytogenes. The term includes glycoproteins, glycopeptides, lipoproteins and lipopeptides. The heterologous polypeptide can be expressed by a host bacterium. In some embodiments, the host bacterium is administered to a cell, e.g., a eukaryotic cell, and the heterologous polypeptide is processed and presented to cytotoxic T-cells. In this embodiment, the heterologous polypeptide expressed by the host bacterium need not have an identical amino acid sequence and/or post-translational modifications as the corresponding polypeptide found in the pathogen or tumor from which the polypeptide is derived. Instead, the polypeptide can be modified from its naturally occurring form to the extent that it still results in a T-cell response that recognizes the polypeptide which is naturally expressed.

“Operably linked” means that expression of the heterologous nucleic acid sequence that is joined to the 5′ UTR is regulated at least to some measurable extent by the 5′ UTR (and/or the promoter, and RBS of the nucleic acid).

The terms “protein” and “polypeptide” are used interchangeably herein.

The details of one or more embodiments of the invention are set forth in the accompanying drawings and the description below. Other features, objects, and advantages of the invention will be apparent from the description and drawings, and from the claims. All cited patents, patent applications, and references (including references to public sequence database entries) are incorporated by reference in their entireties for all purposes.

### DESCRIPTION OF DRAWINGS

FIG. 1 is a table with schematic depictions of various constructs containing hly UTRs and actA UTRs. UTRs were fused downstream of the pHyper promoter and upstream of the ATG start codon of prfA. Dashed lines denote hly 5′ UTR sequences. Solid lines denote actA 5′ UTR sequences. The numbers below each 5′ UTR indicate the nucleotide position of the 5′ UTR relative to the transcriptional start of the native transcript fused to pHyper. Ovals represent the RBS. LLO activity (measured by detecting hemolytic activity of culture supernatants as an indirect measure of PrfA activation of P_{hly} promoter) is shown as hemolytic units and is expressed as the reciprocal of the dilution at which 50% lysis of erythrocytes is observed. The amounts of PrfA protein expressed by each construct (detected by Western blot analysis and expressed as the value given by densitometric analysis relative to the base 20 nucleotide UTR constructs, pHL20SP and pHA20SP) are also shown in the table.

FIG. 2A is a graph showing the effect of deletions of the hly 5′ UTR on the activity of a reporter gene product, Gus. Gus activity was measured 3 hours after 1:10 dilution of overnight cultures in BHI. Units of Gus were normalized for optical density at 600 nm. The constructs used in these experiments are depicted in FIG. 2B.

FIG. 2B is a table showing schematic depictions of constructs containing hly 5′ UTRs fused to gusA and results of experiments in which the constructs were tested. The hly 5′ UTRs were fused directly downstream from the pHyper promoter and upstream of the ATG start codon of gusA. Dashed lines denote hly 5′ UTR. The numbers below each 5′ UTR indicate the nucleotide position of the 5′ UTR relative to the transcriptional start of the native transcript. Gus activity is expressed in enzymatic units (pmol/min*mL*OD600). Values of Gus protein expression were obtained by densitometric analysis. Levels of gus transcript are expressed as the ratio of gus transcript divided by the p60 transcript as detected by phosphorimager analysis.

FIG. 3A is a schematic diagram of nucleic acid constructs containing actA or hly 5′ UTRs and native or ermC RBS which were used to examine expression of a reporter gene product, Gus. ActA and hly 5′ UTRs were fused downstream of the pHyper promoter and upstream of gusA. pHA_{(-rbs)} gus contains the first 130 nt of the actA 5′ UTR fused to gusA such that translation is initiated from the ermC RBS. pHA_{(-rbs)} gus contains the first 113 nucleotides of the hly 5 UTR fused to gusA such that translation initiates from the ermC RBS.

FIG. 3B is a graph showing the effect of exchanging the actA or hly RBS with the ermC RBS. The constructs used for this experiment are depicted in FIG. 3A. Gus activity was measured 3 hours after 1:10-dilution of overnight cultures in BHI. Units of Gus were normalized for optimal density at 600 nm.

### DETAILED DESCRIPTION

The invention is based, in part, on the observation that fusion of all or a part of the Listeria monocytogenes actA or hly 5′ untranslated region (UTR) is sufficient to enhance expression of heterologous nucleic acids. The actA and hly 5′ UTRs can be used to enhance expression of heterologous nucleic acids, for example, in bacteria, e.g., Gram-positive bacteria.

5′ Untranslated Regions

A 5′ untranslated region (5′ UTR) is a nucleic acid sequence located between a promoter sequence and a start codon of a bacterial gene, that is transcribed but not translated. As used herein, a 5′ UTR refers to any sequence 5′ of a bacterial gene that is transcribed but not translated. This includes fragments of nucleic acid sequences that are found between the regulatory and other sequences upstream of the transcriptional start site (e.g., the promoter region) and the start codon of a given gene. Preferably, the 5′ UTRs are derived from the Listeria monocytogenes actA and hly genes. A L. monocytogenes 5′ UTR can be used in association with the ribosome binding site (RBS) native to the UTR, or with a RBS not naturally associated with the specified L. monocytogene. For example, the hly 5′ UTR can be used with the RBS from the hly gene or the hly 5′ UTR can be used with a non-hly gene RBS, e.g., an RBS from a different L. monocytogene or from any other gene. Examples of L. monocytogenes 5′ UTRs and 5′ UTR fragments are provided in the table below. Hly 114-133 and actA 131-150 correspond to 5′ UTR fragments immediately upstream of the hly and actA start codon. The hly and actA ribosome binding sites (RBSs) map within these fragments.

TABLE 1
Exemplary 5′ UTR sequences

SEQ ID

| | | | |
|---|---|---|---|
| Gene | residues | UTR nucleotide sequence | NO: |

| | | | |
|---|---|---|---|
| hly | 1-133 | ATAAAGCAAGCATATAATATTGCGTTTCATCTTTAGAAGCGAATTTCGCCAAT | SEQ ID |
| | | ATTATAATTATCAAAAGAGAGGGGTGGCAAACGGTATTTGGCATTATTAGGTT | NO: 1 |
| | | AAAAAATGTAGAAGGAGAGTGAAACCC | |
| hly | 35-133 | AGAAGCGAATTTCGCCAATATTATAATTATCAAAAGAGAGGGGTGGCAAACGG | SEQ ID |
| | | TATTTGGCATTATTAGGTTAAAAAATGTAGAAGGAGAGTGAAACCC | NO: 2 |
| hly | 69-133 | AGAGAGGGGTGGCAAACGGTATTTGGCATTATTAGGTTAAAAAATGTAGAAGG | SEQ ID |
| | | AGAGTGAAACCC | NO: 3 |
| hly | 114-133 | GTAGAAGGAGAGTGAAACCC | SEQ ID |
| | | | NO: 4 |
| actA | 1-150 | TAATTCATGAATATTTTTTCTTATATTAGCTAATTAAGAAGATAATTAACTGC | SEQ ID |
| | | TAATCCAATTTTTAACGGAATAAATTAGTGAAAATGAAGGCCGAATTTTCCTT | NO: 5 |
| | | GTTCTAAAAAGGTTGTATTAGCGTATCACGAGGAGGGAGTATAA | |
| actA | 52-150 | GCTAATCCAATTTTTAACGGAATAAATTAGTGAAAATGAAGGCCGAATTTTCC | SEQ ID |
| | | TTGTTCTAAAAAGGTTGTATTAGCGTATCACGAGGAGGGAGTATAA | NO: 6 |
| actA | 81-150 | GTGAAAATGAAGGCCGAATTTTCCTTGTTCTAAAAAGGTTGTATTAGCGTATC | SEQ ID |
| | | ACGAGGAGGGAGTATAA | NO: 7 |
| actA | 131-150 | ATCACGAGGAGGGAGTATAA | SEQ ID |
| | | | NO: 8 |

5′ UTR sequences may be used which are homologous, but not identical to the UTR sequences in the Table 1. Functional sequences are sequences that increase expression of an operably linked heterologous nucleic acid sequence relative to the expression of the nucleic acid sequence without the 5′ UTR. 5′ UTR variants can be, for example, at least 50%, 60%, 70%, 80%, 85%, 90%, 95%, 98% or 99% homologous to an actA or hly 5′ UTR or a functional 5′ UTR fragment thereof. In some embodiments, UTR variants may differ from an actA or hly 5′ UTR sequence by more than 1, 2, 3, 4, 5 or 10 nucleotides but less than 25, 35, 45 or 50 nucleotides. In other embodiments, the 5′ UTR variant hybridizes under a stringency condition described herein to an actA or hly 5′ UTR or a functional 5′ UTR fragment thereof. A 5′ UTR variant sequence or 5′ UTR fragment can be assessed for function (e.g., ability to increase expression of a heterologous nucleic acid sequence) using methods described herein.

Calculations of homology or sequence identity between sequences (the terms are used interchangeably herein) are performed as follows.

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. The percent identity between two nucleotide sequences can be determined using the algorithm of Needleman and Wunsch ((1970) J. Mol. Biol. 48: 444-453) algorithm which has been incorporated into the GAP program in the GCG software package, using either a Blossum 62 matrix and a gap weight of 12, a gap extend penalty of 4, and a frameshift gap penalty of 5.

Generally, to determine the percent identity of two nucleic acid sequences, the sequences are aligned for optimal comparison purposes (e.g., gaps can be introduced in one or both of a first and a second nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The nucleotides at corresponding nucleotide positions are then compared. When a position in the first sequence is occupied by the same nucleotide as the corresponding position in the second sequence, then the molecules are identical at that position (as used herein nucleic acid “identity” is equivalent to nucleic acid “homology”).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

Heterologous Nucleic Acid Sequences

Listeria monocytogenes hly and/or actA 5′ UTRs and fragments thereof can be used to enhance the expression of heterologous nucleic acid sequences, e.g., nucleic acid sequences that are not naturally associated with the Listeria monocytogenes 5′ UTR(s) and/or fragments thereof to which they are operably linked. The 5′ UTRs and fragments thereof may be used for expression of a variety of polypeptides for a variety of applications. For example, Listeria monocytogenes 5′ UTRs can be used for production and purification of recombinant proteins.

In one embodiment, the 5′ UTR-nucleic acid construct is part of a system to deliver a polypeptide antigen to MHC molecules of a eukaryotic cell, wherein the heterologous nucleic acid encodes the polypeptide antigen. Polypeptides of interest include viral, fungal, parasitic or bacterial antigens, or tumor-associated antigens. The antigens can be obtained from natural sources or they can be produced by recombinant DNA technology or by other artificial means.

In some embodiments, the 5′ UTR can be used for expression of a nucleic acid encoding a viral polypeptide. For example, the viral polypeptide can be from any human or animal virus to which an immune response is desired, including, but not limited to, the following: human immunodeficiency virus (HIV), hepatitis B virus (HBV), hepatitis C virus (HCV), hepatitis A virus, non-A and non-B hepatitis virus, smallpox, influenza viruses, human papilloma virus, adenovirus, rhinovirus, coronavirus, herpes simplex virus (types I and II), respiratory syncytial viruses, rabies, coxsackie virus, human T-cell leukemia virus (types I, II and III), cytomegalovirus, parainfluenza virus, poliovirus, rotavirus, coronavirus, rubella virus, measles virus, varicella, Epstein Barr virus, adenovirus, and yellow fever virus. The viral polypeptide can be encoded by SARS-CoV. The nucleic acid can also encode a variant of a viral polypeptide, e.g., a known variant of a viral polypeptide, or functional fragments of the viral polypeptide.

Viral antigens can include any viral protein or functional fragment thereof. For example, the nucleoprotein (NP) of influenza virus can be expressed. Examples of HIV antigens include: HIV gag; HIV env or its component parts gp120 and gp41; HIV nef; HIV pol proteins; reverse transcriptase; and HIV protease.

The bacterial antigens of interest include those associated with the human and animal bacterial pathogens including, but not limited to, Mycobacterium spp. (e.g., Mycobacterium tuberculosis, Mycobacterium leprae), Streptococcus spp. (e.g., Streptococcus pneumoniae, Streptococcus pyogenes), Staphylococcus spp. (e.g., Staphylococcus aureus), Treponema (e.g., Treponema pallidum), Chlamydia spp., Vibrio spp. (e.g., Vibrio cholerae), Bacillus spp. (e.g., Bacillus subtilis, Bacillus anthracis), Yersinia spp. (e.g., Yersinia pestis), Neisseria spp. (e.g., Neisseria meningitides, Neisseria gonorrhoeae), Legionella spp., Bordetella spp. (e.g., Bordetella pertussis), Shigella spp., Campylobacter spp., Pseudomonas spp. (e.g., Pseudomonas aeruginosa), Brucella spp., Clostridium spp. (e.g., Clostridium tetani, Clostridium botulinum, Clostridium perfringens), Salmonella spp. (e.g., Salmonella typhi), Borrelia spp. (e.g., Borrelia burgdorferi), Rickettsia spp. (e.g., Rickettsia prowazeki), Mycoplasma spp. (e.g., Mycoplasma pneumoniae), Haemophilus spp. (e.g., Haemophilus influenzae), Branhamella spp. (e.g., Branhamella catarrhalis), Corynebacteria spp. (e.g., Corynebacteria diphtheriae), Klebsiella spp. (e.g., Klebsiella pneumoniae), Escherichia spp. (e.g., Escherichia coli), and Listeria spp. (e.g., Listeria monocytogenes). Functional fragments and variants of polypeptides encoded by such pathogens are known in the art and can be expressed by the 5′ UTR-heterologous nucleic acid constructs described herein.

Fungal polypeptides can be those derived from fungi including but not limited to Candida spp. (e.g., albicans), Cryptococcus spp. (e.g., neoformans), Blastomyces spp. (e.g., dermatitidis), Histoplasma spp. (e.g., capsulatum), Coccidroides spp. (e.g., immitis), Paracoccidroides spp. (e.g., brasiliensis), Aspergillus spp and functional fragments and variants thereof.

Parasitic antigens can be derived from organisms that include, but are not limited to, Plasmodium spp., Eimeria spp., Schistosoma spp., Trypanosoma spp., Babesia spp., Leishmania spp., Cryptosporidia spp., Toxoplasma spp., Pneumocystis spp., Entamoeba histolytica, Giardia spp., Plasmodium spp., Cryptosporidium spp., Trichuris trichura, Trichinella spiralis, Enterobium vermicularis, Ascaris lumbricoides, Ancylostoma spp., Stongyloides spp., Filaria spp., and Schistosoma spp. A parasitic antigen can be an antigen or fragment or variant thereof derived from, e.g., the listed organisms.

Tumor-derived antigens can also be expressed. Exemplary tumor-associated antigens include, but are not limited to, the following: 707 alanine proline (707-AP); alpha (α)-fetoprotein (AFP); adenocarcinoma antigen recognized by T cells 4 (ART-4); B antigen (BAGE); β-catenin/mutated(b-catenin/m); breakpoint cluster region-Abelson (Bcr-abl); CTL-recognized antigen on melanoma (CAMEL); carcinoembryonic antigen peptide-1 (CAP-1); caspase-8 (CASP-8); cell-division cycle 27 mutated (CDC27m); cycline-dependent kinase 4 mutated CDK4/m); carcinoembryonic antigen (CEA); cancer/testis (CT) antigen; cyclophilin B (Cyp-B); differentiation antigen melanoma (DAM-6, also known as MAGE-B2, and DAM-10, also known as MAGE-B1); elongation factor 2 mutated (ELF2M); Ets variant gene 6/acute myeloid leukemia 1 gene ETS (ETV6-AML1); glycoprotein 250 (G250); G antigen (GAGE); N-acetylglucosaminyltransferase V (GnT-V); glycoprotein 100 kD (GnT-V); helicase antigen (HAGE); human epidermal receptor-2/neurological (HER-2/neu); HLA-A*0201-R1701 (HLA-A*0201 having an arginine (R) to isoleucine (I) exchange at residue 170 of the α-helix of the α2-domain in the HLA-A2 gene); human papilloma virus E7 (HPV-E7); human papilloma virus E6 (HPV-E6); heat shock protein 70-2 mutated (HSP70-2M); human signet ring tumor-2 (HST-2); human telomerase reverse transcriptase (hTERT or hTRT); intestinal carboxyl esterase (iCE); KIAA0205; L antigen (LAGE); low density lipid receptor/GDP-L-fucose: β-D-galactosidase 2-α-Lfucosyltransferase (LDLR/FUT); melanoma antigen (MAGE); melanoma antigen recognized by T cells-1/Melanoma antigen A (MART-1/Melan-A); melanocortin 1 receptor (MC1R); myosin mutated (Myosin/m); mucin 1 (MUC 1); melanoma ubiquitous mutated 1 (MUM-1), melanoma ubiquitous mutated 2 (MUM-2), melanoma ubiquitous mutated 3 (MUM-3); New York-esophageous 1 (NY-ESO-1); protein 15 (P15); protein of 190 KD bcr-abl (p190 minor bcr-abl); promyelocytic leukaemia/retinoic acid receptor α (Pml/RARa); preferentially expressed antigen of melanoma (PRAME); prostate-specific antigen (PSA); prostate-specific membrane antigen (PSM); renal antigen (RAGE); renal ubiquitous 1 (RU1), renal ubiquitous 2 (RU2); sarcoma antigen (SAGE); SART-1; SART-3; translocation Ets-family leukemia/acute myeloid leukemia 1 (TEL/AML1); triosephosphate isomerase mutated (TPI/m); tyrosinase related protein 1 (TRP-1 or gp75); tyrosinase related protein 2 (TRP2); TRP-2/intron 2 (TRP-2/INT2); Wilms' tumor gene (WT-1). Preferred tumor-derived antigens include TRP-1, TRP-2, and MART-1/Melan-A.

Bacterial Strains and Expression Systems

The introduction of a nucleic acid encoding a 5′ UTR and the heterologous nucleic acid sequence, e.g., the nucleic acid sequence encoding a polypeptide, into a host bacterium can be accomplished, for example, by the creation of a recombinant bacterium in which a nucleic acid sequence including the 5′ UTR-heterologous nucleic acid is harbored on a vector, such as a plasmid, which is maintained and expressed in the host species. Alternatively, a nucleic acid comprising the 5′ UTR and the heterologous nucleic acid sequence can be stably integrated into the bacterial chromosome by employing, for example, transposon mutagenesis, homologous recombination, or site-specific integration.

Nucleic Acid Constructs

A Listeria monocytogenes actA or hly 5′ UTR can be linked to a nucleic acid sequence, e.g., a nucleic acid encoding a heterologous polypeptide using standard molecular biological techniques. The 5′ UTR-heterologous nucleic acid sequence can be incorporated into a nucleic acid construct that further includes one or more regulatory elements. For example, a promoter can be inserted upstream of the 5′ UTR. Preferably, the promoter is inserted immediately 5′ of the 5′ UTR. Appropriate promoters include those promoters active in the host bacteria being used, e.g., promoters active in Gram-positive bacteria, e.g., a promoter derived from Gram-positive bacteria (see Host Bacteria, below). Constitutive and strong promoters can be used, e.g., to achieve high levels of expression. In other embodiments, the promoter that is naturally associated with the 5′ UTR can be used. Inducible or regulatable promoters can be used, e.g., for expression of proteins that are toxic to the bacterial host cell.

Similarly, the choice of other regulatory elements will depend on the nature of the host bacteria and the method by which the 5′ UTR-heterologous nucleic acid sequence construct will be introduced into the host bacteria. Integration of nucleic acids comprising the 5′ UTR can be performed in a number of ways. For example, phage site-specific integration systems can be used for introduction of constructs into the chromosome of Listeria monocytogenes. Generation of a Listeria containing a chromosomal insertion of a 5′ UTR-heterologous nucleic acid sequence element can be performed as follows. Briefly, the 5′ UTR-heterologous nucleic acid sequence element is first cloned into a shuttle integration vector that contains phage integrase and attachment site (e.g., a U153 listeriophage integrase site). The shuttle integration vector can be propagated in a strain such as E. coli and introduced into the Listeria having the corresponding phage attachment site by conjugation (see, e.g., Lauer et al. J. Bacteriol. 184(15): 4177-4186, 2002). Stable, single copy integration of nucleic acids into Listeria can be achieved in this manner. Transposons can also be used to introduce 5′ UTR-heterologous nucleic acid sequence constructs into the genome of bacteria such as Listeria or B. subtilis. See, e.g., Freitag, Gram-positive Pathogens, Am. Soc. Microbio. p. 488-498, 2000. For strains such as B. subtilis, the nucleic acid can be introduced by homologous recombination (see, e.g., Niaudet B., et al. J. Bacteriol. 163(1): 111-20, 1985). Briefly, 5′ UTR-heterologous nucleic acid sequence constructs can be engineered to include flanking sequences with homology to sequences in the B. subtilis genome. These flanking sequences can recombine with the sequences in the genome, resulting in insertion of the 5′ UTR-heterologous nucleic acid sequence element.

Additional regulatory elements can be used, e.g., to further enhance expression of the nucleic acid of interest. For example, a transcriptional activator sequence can be inserted upstream of the promoter. Expression of Listeria can be enhanced, e.g., by introducing a prfA binding site, or prfA box, upstream of the promoter. Other transcriptional activator sequences can be used. 5′ UTR-heterologous nucleic acid sequence constructs may also be expressed in a host strain using plasmids. For plasmids appropriate for Listeria, see Freitag, Gram-positive Pathogens, Am. Soc. Microbio. p. 488498, 2000. The vector can include other elements as well.

The 5′ UTRs described herein can be used in combination with a B. subtilis bacteriophage SPO-1 promoter. Inducible SPO-1 promoters have been constructed by fusing the SPO-1 promoter to a lac repressor/operator sequence. These include the SPAC/LacO and the SPAC/LacOid promoters (See, e.g., Dancz, CE, et al. J Bacteriol, 184(21): 5935-5945, 2002; GenBank® Acc. No. AY12601).

5′ UTRs can be used in combination with other regulatory elements that allow production of high levels of proteins. For example, the 5′ UTR-heterologous nucleic acid sequences described herein can be used in combination with a bacteriophage T7 RNA polymerase and its cognate promoter. Expression of a target nucleic acid sequence under the control of a bacteriophage T7 promoter in the presence of T7 RNA polymerase results in highly efficient transcription of the target sequence in a bacterial host (Studier, F. W. and Moffatt, B. A. J. Mol. Biol. 189, 113-130, 1986; U.S. Pat. No. 4,952,496). The addition of a 5′ UTR can further optimize expression in combination with these elements. Regulation of expression by a T7 promoter can be achieved by regulating T7 RNA polymerase expression, e.g., by placing T7 RNA polymerase expression under the control of a lac promoter. Various T7 RNA pol expression systems are described, e.g., in the pET System Manual, 10^{th} Ed., 2002 (www.novagen.com). Strategies to optimize expression in Gram-positive hosts include the use of a T7 RNA polymerase system in which T7 polymerase expression is regulated by a inducible promoter native to the Gram-positive host organism. See, e.g., U.S. Pat. No. 6,221,648.

Another regulatory system that can be used in combination with the 5′ UTRs described herein is a system based on the arabinose C (araC) repressor inducer (Lutz R, and Bujard H Nucleic Acids Res. 25(6): 1203-10, 1997).

5′ UTRs can also be used in applications in which enhanced RNA (e.g., mRNA) stability is desired. For example, a 5′ UTR described herein can be incorporated upstream of a nucleic acid that produces a ribozyme when transcribed. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. A ribozyme molecule typically includes one or more sequences complementary to the target gene mRNA, and includes the well known catalytic sequence responsible for mRNA cleavage disclosed, for example, in U.S. Pat. No. 5,093,246. Specific ribozyme cleavage sites within any potential RNA target are initially identified by scanning the molecule of interest for ribozyme cleavage sites that include the sequences GUA, GUU, and GUC. Once identified, short RNA sequences of between 15 and 20 ribonucleotides corresponding to the region of the target gene containing the cleavage site can be evaluated for predicted structural features, such as secondary structure, that may render the oligonucleotide sequence unsuitable. The suitability of candidate sequences can also be evaluated by testing their accessibility to hybridization with complementary oligonucleotides, using ribonuclease protection assays. The 5′ UTRs described herein can be used to enhance stability of other useful RNAs, such as inhibitory or antisense RNAs which can mediate specific inhibition of gene expression.

Host Bacteria

A variety of bacteria can be used for expression of the 5′ UTR-heterologous nucleic acid constructs. Preferably, the host bacterium is nonvirulent and non-pathogenic. In one embodiment, the host bacterium is a Gram-negative bacterium. In another embodiment, the host bacterium is a Gram-positive bacterium. Suitable bacteria include well-characterized, nonvirulent, non-pathogenic strains of Corynebacteria, Listeria spp. (e.g., Listeria monocytogenes), Mycobacteria, Bacillus spp. (e.g., Bacillus subtilis, Bacillus cereus, Bacillus Calmette Guerin), Clostridia, Lactobacillus spp. (e.g., Lactobacillus acidophilus), Staphylococcus (e.g., Staphylococcus epidermidis), Enterococcus spp. (e.g., Enterococcus faecalis), Lactococcus, etc. The choice of bacterium can be determined based upon the desired application. For example, particular strains may be well suited for expression and purification of a recombinant protein. Likewise, particular strains may be appropriate for use as a vaccine.

In one embodiment, the bacteria are attenuated to be nonreplicative, and nonintegrative into the host cell genome, and/or non-motile inter- or intra-cellularly. A variety of suitable means for microbial attenuation are known in the art. For example, the bacteria may be an auxotrophic, attenuated strain of Listeria. See, e.g. U.S. Pat. No. 6,099,848. In another embodiment, the bacteria are non-viable (e.g., alive, but unable to divide) prior to endocytosis by the target cell or administration to the target organism, obviating any microbial growth or metabolism in the target cell. A variety of means for killing or rendering the bacteria non-viable are known in the art. These include fixation with organic solvents such as methanol, UV irradiation, heat, freeze-drying, etc. Some methods preserve the ability of the bacterial membrane and/or wall to retain a cytolysin and a heterologous polypeptide. In one embodiment, a heterologous polypeptide and a cytolysin are sufficiently expressed to load the bacteria with an effective amount of the cytolysin and heterologous polypeptide prior to bacterial cell death. The bacteria can contain (i.e. as a result of expression of a heterologous nucleic acid sequence within the bacteria) from about ten to one thousand, preferably from about one hundred to one thousand cytolysin molecules per bacterium. In one embodiment, the bacteria are treated with 0.5% paraformaldehyde for 30 minutes at approximately 25° C. To calculate dosages of attenuated/inactivated bacteria (e.g., CFU), an aliquot of the bacteria can be obtained prior to the step of inactivation (e.g., fixation) and assayed for growth (e.g., by plating various dilutions on agar plates and calculating CFU). The quantity of bacteria in the attenuated sample can be extrapolated from the results obtained for the viable bacteria.

Cytolysins

Bacteria that express a functional cytolysin can be used for expression of the 5′ UTR-heterologous nucleic acid construct. A variety of cytolysins may be used. Preferably, the cytolysin is not significantly secreted by the bacterium and facilitates cytosolic delivery of the heterologous polypeptide. Bacteria that express non-secreted cytolysins are unable to escape from intracellular compartments of host cells and do not spread to adjacent cells. Functional, non-secreted cytolysins expressed by these cells are released upon degradation of bacteria in intracellular compartments and allow escape of polypeptides (e.g., polypeptides encoded by heterologous nucleic acids) into the cytosol. Cytolysins that can be used include phospholipases (see, e.g., Camilli, A., et al., J. Exp. Med. 173: 751-754, 1991), pore-forming toxins (e.g., an alpha-toxin), natural cytolysins of Gram-positive bacteria, such as listeriolysin O (LLO, e.g. Mengaud, J., et al., Infect. Immun. 56: 766-772, 1988) and Portnoy, et al., Infect. Immun. 60: 2710-2717, 1992), streptolysin O (SLO, e.g. Palmer M, et al., Biochem. 37(8): 2378-2383, 1998) and perfringolysin O (PFO, e.g. Rossjohn J, et al., Cell 89(5): 685-692, 1997). A cytolysin can be selected based on desired physiological properties. For example, when the bacteria are to be administered to phagosomal target cells, acid activated cytolysins can be used. Listeriolysin 0 is a cytolysin that exhibits greater pore-forming ability at mildly acidic pH (the pH conditions within the phagosome), thereby facilitating delivery of the liposome contents to the cytoplasm (see, e.g., Portnoy, et al., Infect. Immun. 60: 2710-2717, 1992). Furthermore, natural cytolysins can be modified to generate mutants (e.g. Awad MM, et al., Microb Pathog. 22(5): 275-284, 1997) having desired activity modifications. Screening assays can be used to measure the ability of a candidate cytolysin to confer on a bacterium the ability to render a target cell vacuole permeable to a label (e.g., a fluorescent or radioactive label) that is contained in the vacuole. See, e.g., U.S. Pat. No. 6,004,815. Alternatively, mutant cytolysins are selected from naturally-occurring mutants by, for example, identifying bacteria which contain cytolysins that are capable of lysing cells over a narrow pH range, preferably the pH range which occurs in phagosomes (pH 5.0-6.0), or under other conditions (e.g., ionic strength) which occur in the targeted phagosomes. Nonsecreted cytolysins may be provided by various mechanisms, including, e.g. absence of a functional signal sequence, a secretion incompetent microbe, such as microbes having genetic lesions (e.g. a functional signal sequence mutation), or poisoned microbes, etc.

The cytolysin can be a cytolysin that is naturally occurring in the bacterial host, or it can be a cytolysin that is introduced by recombinant means. For example, Listeria monocytogenes, which naturally express listeriolysin O, can be used as host bacteria for expression of the 5′ UTR-heterologous nucleic acid construct.

Administration of Bacteria

The bacteria or bacteria-based vaccines of the present invention may be administered to a host vertebrate animal, typically a mammal, (e.g., a human or a mouse), either alone or in combination with a pharmaceutically acceptable carrier. The bacterial composition or bacteria is/are administered in an amount effective to induce an immune or other desired response to the nucleic acid or polypeptide expression product of a 5′ UTR-heterologous nucleic acid construct in a host bacterium.

The bacteria can be used to deliver a nucleic acid or polypeptide product of the heterologous nucleic acid to a target cell. For example, the product nucleic acid or polypeptide can be delivered to a cell capable of endocytosis of the bacteria, including phagocytic, non-phagocytic, pathogenic or diseased cells. Target cell types include epithelial cells, endothelial cells, muscle cells, liver cells, pancreatic cells, neural cells, fibroblasts, tumor cells, leukocytes such as macrophages, neutrophils, B-cells, T-cells, monocytes, etc. For introduction of a nucleic acid or polypeptide product of the heterologous nucleic acid into the cytosol of the target cell, uptake of a bacterium and subsequent lysis within the target cell vacuole (including phagosomes and endosomes) may be desirable. Phagocytic target cells can provide for microbial uptake and lysis. For some cell types, a bacterium with an invasin to facilitate or mediate uptake by the target cell and an autolysin to facilitate or mediate autolysis of the bacterium within the target cell vacuole can be used. A wide variety of suitable invasins and autolysins are known in the art. See, e.g., Sizemore et al. (Science, 270: 299-302, 1995) and Courvalin et al. (C.R. Acad. Sci. Paris, 318: 1207-12, 1995), which disclose expression of an invasin to effect endocytosis of the bacterium by a target cell. Suitable microbial autolysins are described by Cao et al., Infect Immun 66(6): 2984-2986, 1998; Margot et al., J Bacteriol 180(3): 749-752, 1998; Buist et al., App Environ Microbiol, 63(7): 2722-2728, 1997; Yamanaka et al., FEMS Microbiol Lett, 150(2): 269-275, 1997; Romero et al., FEMS Microbiol Lett, 108(1): 87-92; Betzner and Keck, Mol Gen Genet, 219(3), 1989: 489491; Lubitz et al., J. Bacteriol, 159(1): 385-387, 1984; and Tomasz et al., J. Bacteriol, 170(12): 5931-5934, 1988. Delayed lysis can be provided by using temperature-sensitive autolysins, time-sensitive autolysins (see, e.g. Chang et al., J Bact 177, 3283-3294, 1995; Raab et al., J Mol Biol 19, 95-105, 1985; Gerds et al., Mol Microbiol 17, 205-210, 1995) and addiction (poison/antidote) autolysins, (see e.g. Magnuson R, et al., J Biol. Chem. 271(31), 1996, 18705-18710; Smith A S, et al., Mol Microbiol. 26(5), 961-970, 1997).

Administration of bacteria to target cells can be performed in vitro or in vivo. In either case, the methods generally involve growing the bacteria, inducing expression of the heterologous nucleic acid sequence, e.g., such that a polypeptide encoded by the nucleic acid sequence is expressed, and contacting the target cells with an amount of bacteria sufficient to effect the desired activity of the heterologous nucleic acid sequence and/or polypeptide in the target cell(s).

In vitro or ex vivo administration can include contacting the target cell(s) with an effective amount of the bacteria of the invention. In vitro applications include protein delivery (e.g. for functional determinations, toxin delivery to targeted cells in culture, half-life, degradation and localization determinations), and nucleic acid delivery (e.g. RNA to transfected cell lines).

In vivo administration generally can include administering a pharmaceutical composition containing a therapeutically-effective amount of the bacteria of the invention. The therapeutically effective amount will depend on the mode of administration and the strain of bacteria used. Generally, the therapeutically effective amount is an amount of bacteria sufficient to induce an immune or other desired response to a polypeptide or nucleic acid product encoded by the heterologous nucleic acid sequence. For some applications, a therapeutically effective amount of bacteria is an amount of bacteria sufficient to deliver the heterologous nucleic acid sequence and/or a polypeptide or nucleic acid product encoded by the sequence to target cells. In one embodiment, a given number of bacterial cells is administered. Bacteria can be administered as a function of the number of colony forming units (CFU) of the strain. For example, between 1×10³ and 1×10¹¹ CFU of bacteria can be administered per dose. Appropriate dosage ranges include about 1 CFU/kg to about 1×10¹⁰ CFU/kg; e.g., from about 1 CFU/kg to about 1×10⁸ CFU/kg; e.g., 1×10⁴ CFU/kg to about 1×10⁸ CFU/kg. In one embodiment, bacteria are administered orally. See, e.g., Angelakopoulos H, et al. Infect Immun. 70(7): 3592-601 (2002). Briefly, bacteria are cultured, pelleted by centrifugation and washed twice with normal saline. The bacteria are resuspended at a specific turbidity for adminstration in normal saline or a solution that can buffer against gastric acid (e.g., citrate buffer (pH 7.0) containing sucrose; bicarbonate buffer (pH 7.0) alone (Levine et al, J. Clin. Invest., 79: 888-902 (1987); and Black et al J. Infect. Dis., 155: 1260-1265 (1987)), or bicarbonate buffer (pH 7.0) containing ascorbic acid, lactose, and optionally aspartame (Levine et al, Lancet, 11: 467-470 (1988)). Alternatively, a buffer solution is ingested prior to ingestion of the bacteria. The bacteria can be formulated into a pharmaceutical composition by combination with an appropriate pharmaceutically acceptable carrier. Appropriate carriers include proteins, e.g., as found in skim milk, sugars, e.g., sucrose, or polyvinylpyrrolidone. Typically these carriers can be used at a concentration of about 0.1-90% (w/v), and preferably at a range of 1-10% (w/v). The bacteria can be used alone or in appropriate association, as well as in combination with other pharmaceutically active compounds. The bacteria can be administered in combination with an adjuvant. The bacteria can be formulated into preparations in solid, semisolid, or liquid form such as tablets, capsules, powders, granules, ointments, solutions, suppositories, and injections, in usual ways for topical, nasal, oral, parenteral, or surgical administration. Administration in vivo can be oral, mucosal nasal, bronchial, parenteral, subcutaneous, intravenous, intra-arterial, intramuscular, intra-organ, intra-tumoral, or surgical. Administration can include the use of an implantable container (e.g., a biodegradable or semipermeable shell, capsule, tube or other device for delivery of the bacteria) that may optionally contain a matrix upon or into which cells may be seeded. The route of administration can be selected as is appropriate for the targeted host cells. Target cells can also be removed from the subject, treated ex vivo, and the cells then returned to the subject. Other exemplary methods for in vivo administration are described in Shen et al., Proc Natl Acad Sci USA 92(9): 3987-3991, 1995; Jensen et al, Immunol Rev 158: 147-157, 1997; Szalay et al., Proc Natl Acad Sci USA 92(26): 12389-12392, 1995; Belyi et al, FEMS Immunol Med Microbiol 13(3): 211-213, 1996; Frankel et al., J. Immunol 155(10): 4775-4782, 1995; Goossens et al., Int Immunol 7(5): 797-805, 1995; Schafer et al., J. Immunol 149(1): 53-59, 1992; and Linde et al., Vaccine 9(2): 101-105, 1991.

The polypeptide expressed by the administered bacterium may elicit a detectable immune response, e.g., a detectable humoral or cellular immune response. Humoral responses can be detected, for example, by measuring an antibody response to the polypeptide (e.g., serum IgG reactive to the polypeptide, e.g., using an ELISA assay). A cellular immune response is an immune response mediated by T-lymphocytes and/or other white blood cells. The ability of a particular antigen to stimulate a cell-mediated response can be determined by a number of assays, such as by lymphoproliferation assays, CTL cytotoxic cell assays, or by assaying for T-lymphocytes specific for the polypeptide in a subject to which the bacterium has been administered. Such assays are well known in the art. See, e.g., Erickson et al., J. Immunol. (1993) 151: 4189-4199; Doe et al., Eur. J. Immunol. (1994) 24: 2369-2376. T cells that secrete cytokines in response to the polypeptide can be assayed, for example, by ELISPOT analysis (see, e.g., Lalavani, A. R., et al. J. Exp. Med., 186: 859-865, 1997).

Regulation of Listeria Monocytogenes Virulence Gene Expression by PrfA and the Role of 5′ UTRs

Listeria monocytogenes is a facultative intracellular bacterial pathogen that temporally and differentially regulates virulence gene expression in response to specific host cell environments. Expression of two critical virulence genes, hly and actA, increases significantly upon entry into the phagosome and host cytosol, respectively. Hly encodes a pore-forming cytolysin, LLO, required for escape from the phagosome, while actA encodes a surface protein required for nucleating actin polymerization and mediating cell-to-cell spread. Transcription of the actA and hly virulence genes is under the control of PrfA, a transcriptional activator protein belonging to the Crp/Fnr family. PrfA activity, and thus activation of PrfA-dependent promoters, increases upon contact and internalization by host. Although the observed intracellular induction of hly and actA depends upon transcriptional upregulation by PrfA, PrfA-independent factors appear to be required for maximal production of ActA in particular.

Many of the genes required for virulence are located in a 10 kB region known as the PrfA regulon [9]. Expression of most known virulence factors depends upon the transcriptional activator PrfA (encoded by prfA), which activates transcription of virulence gene promoters by binding to palindromic sequences (PrfA boxes) centered at −42 of the promoter region [10, 11, 12]. Of all the known PrfA-regulated genes, only the PrfA box shared by hly and plcA is perfectly symmetrical, while the PrfA boxes preceding the remaining virulence genes have one or two mismatches [13]. Interestingly, only LLO and PI-PLC are produced at high levels during growth in broth, whereas expression of all PrfA-regulated virulence genes is induced during growth in the mammalian cytosol in a temporally and differentially regulated manner [14, 15]. During infection, transcription of genes encoding factors required for vacuolar escape, including hly, precedes transcriptional activation of genes required for intracellular spread, including actA [16]. This differential gene expression has lead to the hypothesis of a “PrfA-site hierarchy” whereby PrfA affinity to PrfA boxes determines virulence gene expression levels [13]. However, recent evidence suggests that the highly regimented virulence gene induction observed during infection cannot be fully attributed to differential activity of PrfA at virulence gene promoters [17, 18].

We have found that fusion of the Listeria monocytogenes actA or hly 5′ UTR is sufficient to enhance expression of two heterologous reporter genes by 5-10 fold. Furthermore, 5′ deletions of actA and hly 5′ UTR fused to these reporter genes results in concomitant decreases in gene expression.

### EXAMPLES

### Example 1

### Regulation of Gene Expression in Bacterial Cells by 5′ UTRs

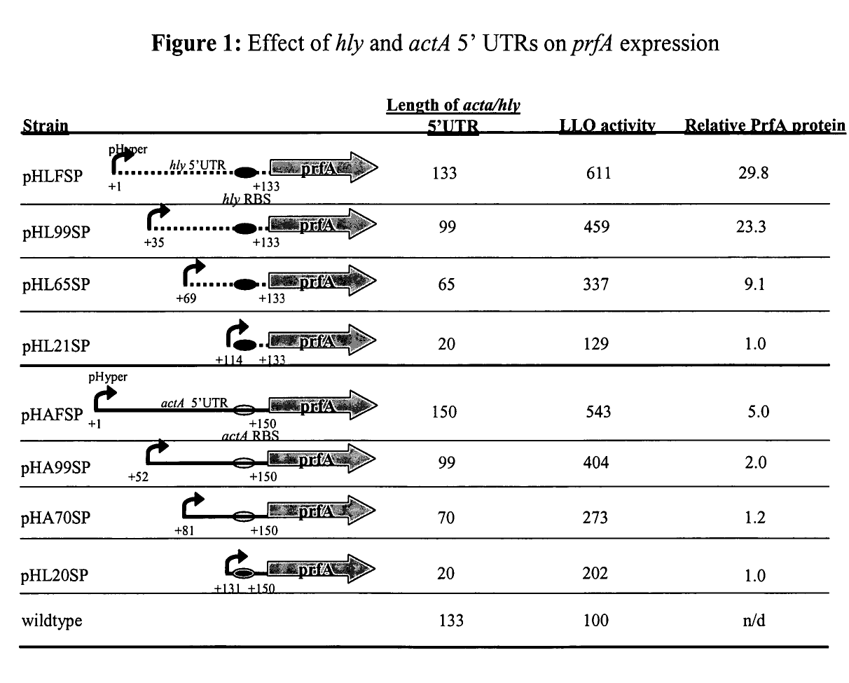

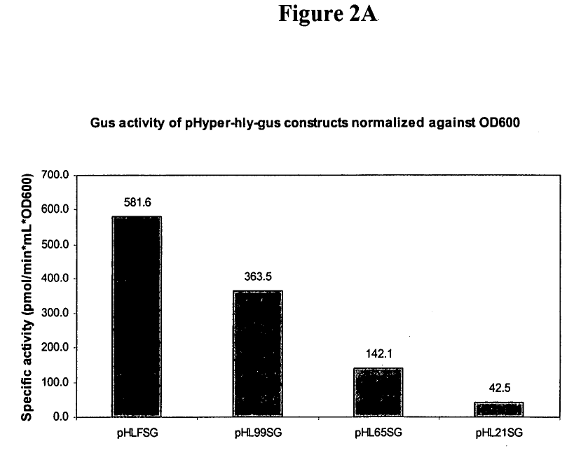

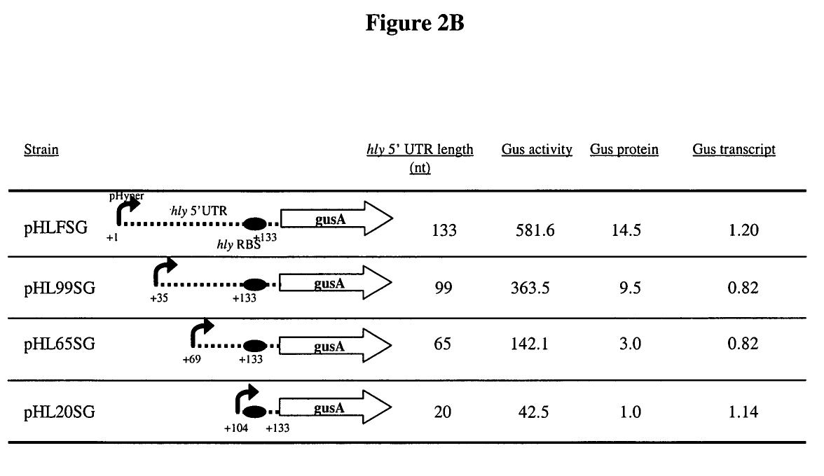

Many Listeria monocytogenes virulence genes have extended 5′ UTRs (greater than 130 nucleotides (nt) in length); nucleic acid modeling programs predict high secondary structure in the 5′ UTRs of these genes. Regulation of gene expression by the 5′ UTRs of actA and hly was examined as follows. Full length actA and hly 5′ UTRs (which contain an RBS; these UTRs correspond to SEQ ID NO:5 and SEQ ID NO:1, respectively) were fused to a reporter gene, prfA, downstream of a modified Bacillus phage SPO-1 promoter (pHyper) in single copy on the chromosome and expressed in L. monocytogenes. PrfA protein levels and PrfA activity expressed by constructs containing these UTRs increased 5-30 fold relative to abbreviated constructs containing only the last 20 nucleotides of these 5′ UTRs (FIG. 1). This enhanced gene expression was not unique to 5′ UTR fusions to prfA, since insertion of actA and hly 5′ UTRs immediately upstream of gusA, an E. coli derived gene that encodes the reporter protein β-glucuronidase, also resulted in increased gene expression in L. monocytogenes (FIGS. 2A and 2B). Furthermore, 5′ deletions of actA and hly 5′ UTRs correlated with decreased expression of reporter genes fused to these 5′ UTRs, indicating that these non-coding sequences function in modulating gene expression.

Primer extension analyses and promoterless fusions of the hly and actA 5′ UTR to prfA and gusA indicated that the inserted 5′ UTR does not introduce additional promoters, suggesting that increased transcription from pHyper is not responsible for the observed increase in gene expression. Fusion of 99 nucleotides of the pdhA coding region, a sequence with little secondary structure, or the trp attenuator, a sequence with considerable secondary structure, to the gusA reporter gene failed to enhance Gus production. Thus, specific sequences within the actA and hly 5′ UTRs mediate enhanced gene expression. The 5′ UTR sequences alone are sufficient, since they confer increased gene expression to two reporter genes from distantly related organisms.

### Example 2

### Regulation of Gene Expression by 5′ UTRs in Mammalian Host Cells

Like many bacterial intracellular pathogens, L. monocytogenes preferentially expresses specific genes within host cells relative to growth in broth: upon entry into host cells, the bacterium specifically induces transcription of virulence genes in a compartment-regulated manner [14, 16, 20]. Indeed, with the exception of hly and plcA, expression of most virulence determinants is low to undetectable during growth in broth. Enhanced expression can be observed in bacteria that have entered host cells. For example, when β-galactosidase was used as a reporter gene to measure hly and actA promoter activity, transcription of actA increased 150 to 200-fold during growth in the mammalian cell cytosol relative to growth in brain heart infusion (BHI). Under these same conditions, hly transcription increased by 20-fold [20]. This same study demonstrated that actA is transcribed three-fold more than hly during intracellular growth in macrophage-like cells. This modest increase in transcription, however, results in a 70-fold increase in ActA protein relative to LLO, as determined by quantitative immunoprecipitation.

Increases in transcription and protein expression in bacteria that enter host cells, such as L. monocytogenes bacteria that enter macrophages, can be determined by comparing growth in broth vs. growth in the host cells as described above. Suitability of bacterial strains for use with the 5′ UTRs described herein can be tested by determining the degree to which the 5′ UTR can mediate increases in nucleic acid expression upon entry into host cells as compared to a reference strain (e.g., a Listeria monocytogenes strain).

As described in Example 1, protein production by strains expressing pHyper-5′ UTR-reporter genes depends on the length of the actA and hly 5′ UTRs. The differential gene expression observed between the strains containing deleted 5′ UTRs may depend on post-transcriptional mechanisms of gene regulation. Transcriptional initiation from pHyper is presumably equivalent between these deletion strains, and primer extension analysis indicates that the 5′ UTR insertions do not introduce secondary promoters. These analyses also indicate that for pHyper-hly-gus constructs, steady-state gus transcript levels appeared unaffected by hly 5′ UTR length; although slight differences were observed in steady-state transcripts between constructs, they cannot account for the observed large differences in Gus activity. This result suggests that stabilization of the gus transcript by the hly 5′ UTR does not mediate enhanced gene expression. However, actA and hly transcripts have unusually long half-lives (approximately 30′) when bacteria are grown in minimal essential medium [21], a condition that induces synthesis of many PrfA-regulated proteins but does not support bacterial growth. Thus, the actA and hly 5′ UTRs may confer increased stability to their transcripts, resulting in the high levels of cytosolic LLO and ActA observed in the mammalian cytosol.

### Example 3

### Evaluation of Candidate 5′ UTR Sequences

Regions of 5′ UTR sequences useful for increasing expression of heterologous nucleic acids can be determined, e.g., by mutation and/or deletion of Listeria monocytogenes 5′ UTR sequences (e.g., Listeria monocytogenes actA or hly 5′ UTR sequences), followed by cloning of the sequences downstream of a promoter and upstream of a reporter gene (also described in more detail below). The 5′ UTR sequence includes an RBS or is placed upstream of an RBS. Reporter gene expression can be assayed and levels of gene expression can be compared to levels of expression of a reporter gene lacking the modified 5′ UTR sequences and/or a reporter gene fused to a minimal 5′ UTR sequence that does not mediate enhanced expression. Mutations and deletions that reduce or increase expression can be identified. 5′ UTR sequences associated with genes other than L. monocytogenes actA and hly can also be examined using reporter gene assays, e.g., as described herein.

Evaluation of Native Gene Expression

Deletions in the 5′ UTRs of PrfA-regulated actA and hly (e.g., deletions defined in Example 1 using gusA and prfA reporter genes) can be constructed to assess regulation of reporter gene expression and native gene expression by these 5′ UTRs. Functional fragments and variants of 5′ UTRs for use with heterologous nucleic acid sequences can be evaluated in the context of native gene expression.

One exemplary method of testing regulation of native gene expression by Listeria monocytogenes 5′ UTRs containing a deletion is performed as follows. The 5′ UTR deletions are constructed using the gene splicing by overlap extension (SOEing) method of PCR; truncated hly and actA 5′ UTRs are ligated into the site-specific shuttle integration vector, pPL2, a suicide plasmid that integrates into the 3′end of the tArg gene at a unique site on the chromosome of L. monocytogenes [22].

To test the function of the 5′ UTRs in regulation of native gene expression, second-site complementation of an hly deletion strain (Δhly) and a prfA deletion strain is performed using the pPL2 integration vector [23]. The complete hly structural gene and truncated hly genes with 5′ UTR deletions are cloned into pPL2 and conjugated from an E. coli donor strain into DPL-2161. The actA gene, containing promoters from both mpl and actA genes (Pₘₚₗ and P_{actA}), are amplified from an mpl deletion strain to allow actA expression from both PrfA-regulated promoters; deletions in the actA 5′ UTR are constructed by PCR SOEing. These actA constructs are then cloned into pPL2 and conjugated from E. coli into an actA deletion strain (ΔactA). ActA and LLO levels in strains harboring 5′ UTR deletions are examined during growth in broth, and/or by Western analysis; the effect of 5′ UTR deletions on intracellular growth and spread is measured by plaquing analysis and compared to second-site complementation strains and 10403S. The L2 plaquing assay directly measures cell-to-cell spread and indirectly measures ActA and LLO levels, as decreases in either protein during intracellular growth manifests as a decrease in cell-to-cell spread [18, 23].

Evaluation of Reporter Gene Expression

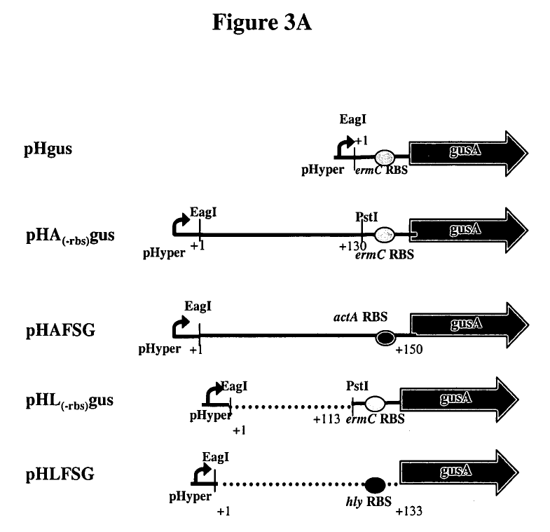

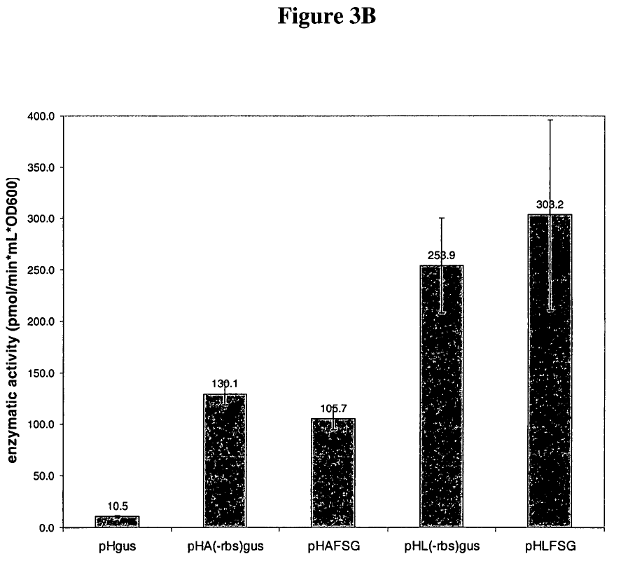

Functional fragments and variants of 5′ UTRs for use with heterologous nucleic acid sequences can be evaluated in the context of reporter gene expression. To identify residues or regions in the 5′ UTRs of actA and hly required for enhancing reporter gene expression, the 5′ UTRs of actA and hly fused to the gusA reporter are subjected to random PCR mutagenesis: mutations that decrease gene expression are readily identified as those that produce light blue or white colonies on 5-bromo-4-chloro-3-indoyl-β-D-glucuronide (X-glu) plates. To minimize the isolation of mutants harboring mutations in the RBS, the last 20 nucleotides of the 5′ UTR are not subjected to PCR mutagenesis; translation of gusA is initiated by the ermC RBS, which is optimized for expression in Gram-positive organisms. This screening strategy excludes the native actA or hly RBS from participating in 5′ UTR 2° structure formation or enhanced protein production and introduces a PstI restriction enzyme site at the 3′ end of the truncated 5′ UTR that may alter natural folding of the 5′ UTR. However, replacement of the last 20 nt of either the actA or hly 5′ UTRs with the ermC RBS had little effect on gusA expression in L. monocytogenes (FIGS. 3A, B), indicating that this screening strategy can allow identification of 5′ UTR residues required to enhance gene expression.

Evaluation of 5′ UTRs Containing Point Mutations

While mutations in the actA and hly 5′ UTRs that increase or decrease gene expression can be defined in the context of their native genes, other screening methods can be used, e.g., methods that employ reporter genes, prior to confirming the activity of the mutant 5′ UTRs on regulation of native gene expression. ActA and hly 5′ UTR mutations that increase or decrease gusA reporter gene expression can be introduced into the 5′ UTR region of their native PrfA-regulated genes in their native context in the PrfA regulon by allelic exchange. Since chromosomal alteration by allelic exchange is a time-consuming process, site-directed mutagenesis of pPL2 constructs containing complete hly and actA genes can also be used to introduce mutations in their respective 5′ UTRs. Expression levels of the site-specific actA and hly 5′ UTR mutants can be compared against wildtype (10403S), the appropriate deletion strains (ΔactA and Δhly), and second-site complemented ΔactA and Δhly strains. Promoterless actA and hly genes can also be placed under the heterologous control of a promoter such as pHyper and cloned into pPL2 and; plasmids containing pHyper-driven actA (pH-actA) and pHyper-driven hly (pH-hly) are be transformed into ΔactA and Δhly, respectively. While these strains are not required to directly test the role of 5′ UTRs in gene expression, they can allow high levels of actA and hly expression independent of PrfA and direct comparison of ActA and LLO production under different environmental conditions.

If specific sequences within the actA and hly 5′ UTRs are essential to mediate enhanced reporter gene expression, point mutations in their 5′ UTRs that alter Gus activity on X-Glu indicator plates can be identified. Providing that these 5′ UTRs function similarly in regulating expression of their native genes, introduction of the mutations that decrease gus expression will likewise diminish expression of these virulence genes both in broth, as detected by reduced protein levels, and in the cytosol, as detected by diminished or absent plaquing in L2 cells relative to 10403S. However, the plaquing assay may give low resolution of ActA and LLO levels. Thus, direct measurement of protein levels in mammalian cells may be required to detect small, but significant, changes in the amount of protein made during intracellular growth between 5′ UTR mutants relative to wildtype 5′ UTR constructs.

To identify 5′ UTR mutations that increase or decrease nucleic acid expression exclusively in the cytosol, deletion strains may be used. For example, such mutations may be identified using actA and hly 5′ UTR fusions to the reporter gene gfp-mut2 expressed in the ΔactA strain. Since ΔactA is defective in cell-to-cell spread, J774 cells infected with this mutant exhibit higher localized fluorescence relative to infection with wildtype Listeria; thus, mutations in the actA and hly 5′ UTRs that reduce gfp expression in ΔactA during growth in the mammalian cytosol should be readily detected and isolated using fluorescence-activated cell sorting. However, this strategy does not account for the role of downstream coding sequences of actA and hly in regulating the ability of their 5′ UTRs to modulate gene expression. To investigate this, strains harboring deletions in the actA and hly 5′ UTR can allow identification of candidate regions for site-directed mutagenesis of these 5′ UTRs in the context of their native genes.

Equivalent increases or decreases in ActA and LLO levels are expected for strains expressing actA and hly harboring 5′ UTR mutations from either the native or tArg locus, provided that full complementation is observed for hly and actA integrated into the tArg locus. Second-site complementation of ΔactA from the tArg locus may not be observed, since second-site complementation by actA expressed from P_{actA} fails to produce wildtype plaques in L2 cells [22].

Mutations introduced into the 5′ UTRs of actA and hly constitutively expressed from the pHyper promoter may increase or decrease ActA and LLO production, respectively. Construction of these strains, however, may be difficult, since construction requires cloning steps in E. coli, which are sensitive to low-level expression of ActA and LLO from pHyper. Attempts to integrate hly driven by pHyper into the tArg locus of Δhly have so far been unsuccessful, perhaps due to overexpression of hly from this strong, constitutive promoter in L. monocytogenes. Expression of actA and hly from pHyper may require that these genes be cloned downstream of an inducible pHyper-promoter that contains the lacOid sequence immediately downstream of the transcriptional start site. The 5′ UTR mutations introduced by allelic exchange may allow assessment of the significance of the actA and hly 5′ UTRs in expression of their native genes.

### Example 4

### Evaluating Regulation of Protein Production by the actA and hly 5′ UTRs in Mammalian Host Cells

Although ActA is induced 70-fold more than LLO in the cytosol of J774 cells [20], PrfA-dependent transcription of actA during intracellular growth accounts for less than 1/20 of the observed increase. Indeed, PrfA-independent mechanisms appear to be involved in the preferential induction of ActA in the mammalian cytosol. Preliminary studies that the actA 5′ UTR may play a role in regulating ActA levels in the cytosol, since select 20 nt deletions in the actA 5′ UTR decrease plaque size in L2 cells (N. Frietag pers. communication). Based on these observations, the actA 5′ UTR may function in specifically inducing actA expression in the cytosol such that ActA is present at considerably higher levels than LLO. In contrast with ActA, LLO is induced only moderately (20-fold) upon entry into the cytosol. This induction may result from increased transcriptional activation of P_{hly} by PrfA. To assess the role of the hly and actA 5′ UTRs on gene expression in the cytosol of a mammalian host cell, the effect of 5′ UTR mutations in hly and actA on the absolute levels of LLO and ActA protein produced during growth in the mammalian cytosol and in broth can be compared. Comparison of protein levels in these two environments, however, is complicated by the responsiveness of PrfA activity to environmental conditions, given that PrfA activity increases upon entry into the cytosol [15, 18, 21]. To uncouple actA and hly expression from PrfA-dependent activation, the pH-actA and pH-hly strains can be used to compare absolute ActA and LLO levels produced in these two disparate environments.

Induction of Protein Expression in the Mammalian Cytosol by the hly 5′ UTR

To evaluate regulation of gene expression by the hly 5′ UTR in mammalian cytosol, expression of the gene product naturally regulated by the hly 5′ UTR, LLO, can be examined. For example, the amount of LLO protein produced by pH-hly and 5′ UTR pH-hly mutants during growth in broth can be compared to the amount produced in the cytosol. Wildtype L. monocytogenes can be used as a positive control, while Δhly can be used as a negative control. For LLO measurements in bacteria growing in broth, mid-log cultures growing at 37° C. in BHI are be pulsed with ³⁵S-Met for 30′, after which samples are be removed for CFU determination and preparation of protein samples. The supernatants of broth grown cultures are TCA precipitated to isolate metabolically labeled LLO. For LLO measurements in bacteria growing intracellularly, J774 cells are be infected with 10403S, Δhly, pH-hly, 5′ UTR pH-hly mutants in triplicate at an MOI of 1:1 for 30′. The monolayers are then be washed; 30′ later, gentamicin is added to kill extracellular bacteria. At 3.5 hr post-infection, the media is replaced with DME without methionine supplemented with cycloheximide and anisomycin, to inhibit the addition of DME without methionine, one monolayer is be pulse-labeled for 30′ with ³⁵S-Met, while cold Met is added to the remaining monolayers; all monolayers are then washed and lysed to release intracellularly growing bacteria. For CFU determination, bacteria are pelleted from the J774 lysate prepared from the monolayer to which cold Met was added. Immunoprecipitation (IP) of one of the labeled J774 lysates is performed using an antibody directed against LLO. Protein samples isolated from broth grown cultures and infected J774 cells are normalized for CFU and resolved by SDS-PAGE; the amount of detectable LLO is quantitated by phosphorimaging.

To determine the efficiency of IP, in vitro labeled LLO (from broth grown cultures) is added to the remaining cold J774 lysate; labeled LLO is then immunoprecitated from this spiked lysate. The immunoprecipitated labeled LLO and a sample of the in vitro labeled LLO used to spike the lysate is subjected to SDS-PAGE and phosphorimaging analysis. The efficiency of IP can be determined by dividing the value obtained by phosphorimaging analysis of immunoprecipitated LLO to that of total LLO (in vitro labeled LLO sample).

Induction of Protein Expression in the Mammalian Cytosol by the actA 5′ UTR

To evaluate regulation of gene expression by the actA 5′ UTR in mammalian cytosol, induction of ActA expression can be studied. For example, the amount of ActA protein produced by pH-hly and 5′ UTR pH-actA mutants during growth in broth can be compared to the amount produced in the cytosol. Samples are prepared as described for LLO except that protein samples isolated from broth culture are boiled in SDS and the resulting supernatant enriched in surface-associated proteins is loaded. Detection of ActA produced in the cytosol can be performed as described for LLO except samples is not immunoprecipitated; instead, the entire J774 lysate is subjected to SDS-PAGE and phosphorimaging analysis.

Since induction of ActA appears to occur by PrfA-independent mechanisms and requires an intact 5′ UTR, the actA 5′ UTR may be sufficient to confer enhanced gene expression. Induction of gene expression in mammalian cytosol by the actA 5′ UTR and fragments and mutant forms thereof can be examined using a reporter gene, such as gusA. The Gus activity of pHAFSG and strains harboring actA 5′ UTR mutations fused to gusA during growth in the cytosol can be compared to Gus production in broth culture. A strain containing gusA inserted between actA and plcB in the PrfA regulon (NF-L476) can be used as a positive control for Gus induction in the cytosol, since Gus activity/CFU of this strain increases 150-fold during growth in J774 cells. The Gus activity of gusA driven by pHyper alone (pH-gus) in these two environments can also be compared and can serve as a control strain that should exhibit no cytosolic induction. Gus activity measurements in broth can be taken from mid-log cells and normalized against CFU, while measurements in infected J774 host cells can be taken 6 hr post-infection and normalized against CFU.

Although actA 5′ UTR function may be altered when fused to a heterologous gene, the assay described above can allow easier and more accurate comparison of gene expression in the cytosol relative to broth, since Gus is not secreted, and so, is protected from host proteases, unlike LLO. This experiment also allows the constitutive nature of pHyper to be tested, since the Gus activity measured for pHyper-gus should be independent of growth conditions.

### Example 5

### Evaluating Stability and Translation Efficiency of Transcripts Containing 5′ UTRs

Listeria monocytogenes 5′ UTRs may affect gene expression by modulating transcript stability and/or translation efficiency. The stability and translation efficiency of transcripts linked to 5′ UTRs can be determined, e.g., to evaluate 5′ UTRs, fragments, and variants thereof. Assays to determine transcript stability and translation efficiency of native actA and hly can be performed using constructs described herein as follows. Transcript and protein levels of pH-actA and pH-hly following rifampicin treatment are compared to pH-actA and pH-hly strains, wherein actA and/or hly is operably linked to a 5′ UTR, fragment, or variant thereof. Wildtype L. monocytogenes are used as a positive control, while Δhly and ΔactA are used as negative controls for LLO and ActA, respectively. These measurements are performed on bacteria grown in broth and in the cytosol, providing that the actA and hly 5′ UTRs function in inducing gene expression in the cytosol. For broth-grown cultures, bacteria in early to mid-log are treated with 100 μg/mL rifampicin and samples for RNA isolation removed 2′, 5′, 10′, 20′, and 40′ after treatment; transcript levels are measured by primer extension. To measure de novo protein synthesis following rifampicin addition, ³⁵S-Met is added 2′, 5′ 10′, 20′, and 40′ after rifampicin treatment. Cells are pelleted 5′ following ³⁵S-Met addition, washed once, and surface associated proteins are extracted by boiling; supernatants are TCA-precipitated. Labeled proteins are detected by SDS-PAGE fluorography and quantitated by phosphorimaging analysis.

For bacteria growing intracellularly, the same infection protocol described above can be used with the following modifications: rifampicin is added 30′ after addition of the eukaryotic protein synthesis inhibitors; RNA is harvested at 5′, 10′, 20′, and 40′ following rifampicin addition. ³⁵S-Met will be added 5′, 10′, 20′, and 40′ after rifampicin addition to measure de novo protein synthesis; 30′ after label is added, J774 cells are lysed for determination of CFU and protein sample preparation. Labeled proteins are resolved by SDS-PAGE and quantified using phosphorimager analysis. Transcript levels are quantitated using real-time RT-PCR, which allows more sensitive quantitation of transcript levels from small amounts of RNA. As a control to allow direct comparison of transcript levels between RNA samples, RT-PCR can be simultaneously be performed on the 16 rRNA gene.

If the actA and hly 5′ UTRs enhance gene expression by stabilizing their respective transcripts, the half-lives of these transcripts should decrease for strains expressing mutant 5′ UTRs that decrease ActA or LLO production. Likewise, if the actA and hly 5′ UTRs confer increased translational efficiency to their native genes then a decrease in labeled ActA and LLO, which does not correlate with a decrease in actA and hly transcript, should be observed in 5′ UTR mutant strains relative to strains with wildtype 5′ UTRs following rifampicin treatment. The 5′ UTRs of actA and hly may both stabilize and increase the translation of their respective transcripts. Should this dual mechanism be responsible for increasing gene expression, a decrease in transcript stability and a concomitant decrease in ribosomes associated with these transcripts will be observed in 5′ UTR variants relative to strains with wildtype 5′ UTRs when cells are treated with rifampicin and fusidic acid, an antibiotic that traps translating ribosomes on transcripts. Interpretation of results will be difficult if rifampicin alone inhibits production of a factor required to either stabilize or increase translation efficiency such that no difference in transcript stability or protein production is observed between strains with wildtype 5′ UTRs or 5′ UTR mutations. Alternatively, it may also be possible to isolate 5′ UTR mutations that affect transcript stability whereas other mutations affect translation efficiency, unless these two mechanisms of post-transcriptional regulation are inseparable.

Evaluating 5′ UTR Secondary Structure

The secondary structure of a 5′ UTR may determine its ability to modulate gene expression. Mutations in 5′ UTRs that increase or decrease expression of linked nucleic acids may alter the secondary structure of the UTR. Compensatory mutations, which restore normal basepairing, would be expected to form secondary structures similar to wildtype sequences. Secondary structure of 5′ UTRs can be determined by chemical probing.

Other extragenic factors may contribute to regulation of gene expression by 5′ UTRs; these factors can be identified using a transposon mutagenesis screen for decreased expression of a reporter gene fused to the 5′ UTR (e.g., using a 5′ UTR-gusA fusion).
1. Vazquez-Boland, J. A., et al., Listeria pathogenesis and molecular virulence determinants. Clin. Microbiol. Rev., 2001. 14(3): p. 584-640.
2. Dramsi, S., Kocks, C., Forestier, C., and Cossart, P., Internalin-mediated invasion of epithelial cells by Listeria monocytogenes is regulated by the bacterial growth state, temperature, and the pleiotropic activator prfA. Molecular Microbiology, 1993. 9(5): p. 931.
3. Domann, E., et al., Detection of a prfA-independent promoter responsible for listeriolysin gene expression in mutant Listeria monocytogenes strains lacking the PrfA regulator. Infection and Immunity, 1993. 61(7): p. 3073-5.
4. Camilli, A., L. G. Tilney, and D. A. Portnoy, Dual roles of plcA in Listeria monocytogenes pathogenesis. Mol. Microbiol., 1993. 8: p. 143-157.
5. Marquis, H., H. Goldfine, and D. A. Portnoy, Proteolytic pathways of activation and degradation of a bacterial phospholipase C during intracellular infection by Listeria monocytogenes. J. Cell Biol., 1997. 137(6): p. 1381-1392.
6. Chico-Calero, I., et al., Hpt, a bacterial homolog of the microsomal glucose-6-phosphate translocase, mediates rapid intracellular proliferation in Listeria. Proc. Natl. Acad. Sci. U.S.A., 2002. 99(1): p. 431-436.
7. Brundage, R. A., et al., Expression and phosphorylation of the Listeria monocytogenes ActA protein in mammalian cells. Proc. Natl. Acad. Sci. U.S.A., 1993. 90: p. 11890-11894.
8. Marquis, H., V. Doshi, and D. A. Portnoy, The broad-range phospholipase C and a metalloprotease mediate listeriolysin O-independent escape of Listeria monocytogenes from a primary vacuole in human epithelial cells. Infect. Immun., 1995. 63(11): p. 4531-4534.
9. Kreft, J. and J. A. Vazquez-Boland, Regulation of virulence genes in Listeria. Int. J. Med. Microbiol., 2001. 291(2): p. 145-157.
10. Chakraborty, T., et al., Coordinate regulation of virulence genes in L. monocytogenes requires the product of the prfA gene. J. Bacteriol., 1992. 174: p. 568-574.
11. Freitag, N. E., L. Rong, and D. A. Portnoy, Regulation of the prfA transcriptional activator of Listeria monocytogenes: multiple promoter elements contribute to intracellular growth and cell-to-cell spread. Infection and Immunity, 1993. 61(6): p. 2537-44.
12. Williams J. R., T., C., Frietag, N. F., Seqence Variations within PrfA DNA Binding Sites and Effects on Listeria monocytogenes virulence gene expression. Journal of Bacteriology, 2000. 182(3): p. 837-841.
13. Sheehan, B., Klarsfeld, A., Msadek, T., Cossart, P., Differential Activation of Virulence Gene Expression by PrfA, the Listeria monocytogenes Virulence Regulato. Journal of Bacteriology, 1995. 177(22): p. 6469-6476.
14. Frietag, N. E. a. J., K. E., Examination of Listeria monocytogenes Intracellular Gene Expression by Using the Green Fluorescent Protein of Aequorea victoria. Infection & Immunity, 1999. 67(4): p. 1844-1852.
15. Renzoni, A., Cossart, P., and Dramsi, S., PrfA, the transcriptional activator of virulence genes, is upregulated during interaction of Listeria monocytogenes with mammalian cells and in eukaryotic cell extracts. Molecular Microbiology, 1999. 34(3): p. 552-561.
16. Bubert, A., et al., Differential expression of Listeria monocytogenes virulence genes in mammalian host cells. Mol. Gen. Genet., 1999. 261(2): p. 323-336.
17. Greene, S. L. a. F., N. E., Negative regulation of PrfA, the key activator of Listeria monocytogenes virulence gene expression, is dispensable for bacterial pathogenesis. Microbiology, 2003. 149: p. 111-120.
18. Shetron-Rama, L. M., et al., Intracellular induction of Listeria monocytogenes actA expression. Infect. Immun., 2002. 70(3): p. 1087-1096.
19. Johansson, J., Mandin, P., Renzoni, A., Chiarutini, C., Springer, M., and Cossart, P., An RNA Thermosensor Controls Expression of Virulence Genes in Listeria monocytogenes. Cell, 2002. 110: p. 551-561.
20. Moors, M. A., et al., Expression of listeriolysin O and ActA by intracellular and extracellular Listeria monocytogenes. Infect. Immun., 1999. 67(1): p. 131-139.
21. Bohne, J., Z. Sokolovic, and W. Goebel, Transcriptional regulation of prfA and PrfA-regulated virulence genes in Listeria monocytogenes. Molecular Microbiology, 1994. 11(6): p. 1141-50.
22. Lauer, P., et al., Construction, characterization, and use of two Listeria monocytogenes site-specific phage integration vectors. J. Bacteriol., 2002. 184(15): p. 4177-4186.
23. Dancz, C. E., et al., Inducible control of virulence gene expression in Listeria monocytogenes: temporal requirement of listeriolysin O during intracellular infection. J. Bacteriol., 2002. 184(21): p. 5935-5945.

A number of embodiments of the invention have been described. Nevertheless, it will be understood that various modifications may be made without departing from the spirit and scope of the invention. Accordingly, other embodiments are within the scope of the following claims.

1. An isolated nucleic acid comprising:
a 5′ untranslated region (UTR), wherein the 5′ UTR comprises a Listeria monocytogenes 5′ UTR selected from the group consisting of a Listeria monocytogenes hly 5′ UTR, a Listeria monocytogenes actA 5′ UTR, and functional fragments and variants thereof; a ribosome binding site (RBS); a heterologous nucleic acid sequence wherein the UTR is operably linked to the heterologous nucleic acid sequence.

2. The nucleic acid of claim 1, wherein the nucleic acid further comprises a promoter.

3. The nucleic acid of claim 2, wherein the nucleic acid further comprises a transcriptional activation site 5′ of the promoter.

4. The nucleic acid of claim 3, wherein the transcriptional activation site is a prfA box.

5. The nucleic acid of claim 1, wherein the RBS is the RBS that is naturally associated with the Listeria monocytogenes UTR.

6. The nucleic acid of claim 1, wherein the Listeria monocytogenes 5′ UTR is the hly 5′ UTR or a functional fragment or variant thereof.

7. The nucleic acid of claim 6, wherein the 5′ UTR comprises a nucleotide sequence at least 70% homologous to the following sequence:

| | |
|---|---|
| AGAGAGGGGTGGCAAACGGTATTTGGCATTATTAGGTTAAAAAATGTAGAAGGAGAGTGAAAC | (SEQ ID NO: 3) |
| CC. | |

8. The nucleic acid of claim 6, wherein the 5′ UTR comprises a nucleotide sequence at least 70% homologous to the following sequence:

| | |
|---|---|
| AGAAGCGAATTTCGCCAATATTATAATTATCAAAAGAGAGGGGTGGCAAACGGTATTTGGCAT | (SEQ ID NO: 2) |
| TATTAGGTTAAAAAATGTAGAAGGAGAGTGAAACCC. | |

9. The nucleic acid of claim 6, wherein the hly 5′ UTR comprises a nucleotide sequence at least 70% homologous to the following sequence:

| | |
|---|---|
| ATAAAGCAAGCATATAATATTGCGTTTCATCTTTAGAAGCGAATTTCGCCAATATTATAATTA | (SEQ ID NO: 1) |
| TCAAAAGAGAGGGGTGGCAACGGTATTTGGCATTATTAGGTTAAAAAATGTAGAAGGAGAGTGAAACC | |
| C. | |

10. The nucleic acid of claim 1, wherein the Listeria monocytogenes 5′ UTR is the actA 5′ UTR or a functional fragment or variant thereof.

11. The nucleic acid of claim 10, wherein the 5′ UTR comprises a nucleotide sequence at least 70% homologous to the following sequence:

| | |
|---|---|
| GTGAAAATGAAGGCCGAATTTTCCTTGTTCTAAAAAGGTTGTATTAGCGTATCACGAGGAGG | (SEQ ID NO: 7) |
| GAGTATAA. | |

12. The nucleic acid of claim 10, wherein the 5′ UTR comprises a nucleotide sequence at least 70% homologous to the following sequence:

| | |
|---|---|
| GCTAATCCAATTTTTAACGGAATAAATTAGTGAAAATGAAGGCCGAATTTTCCTTGTTCTAA | (SEQ ID NO: 6) |
| AAAGGTTGTATTAGCGTATCACGAGGAGGGAGTATAA. | |

13. The nucleic acid of claim 10, wherein the actA 5′ UTR comprises a nucleotide sequence at least 70% homologous to the following sequence:

| | |
|---|---|
| TAATTCATGAATATTTTTTCTTATATTAGCTAATTAAGAAGATAATTAACTGCTAATCCAAT | (SEQ ID NO: 5) |
| TTTTAACGGAATAAATTAGTGAAAATGAAGGCCGAATTTTCCTTGTTCTAAAAAGGTTGTATTAGCGT | |
| ATCACGAGGAGGGAGTATAA. | |

14. The nucleic acid of claim 1, wherein the nucleic acid comprises an integration site.

15. The nucleic acid of claim 1, wherein the heterologous nucleic acid sequence encodes a viral polypeptide or an antigenic fragment thereof.

16. The nucleic acid of any of claim 1, wherein the heterologous nucleic acid encodes an inhibitory RNA or portion thereof.

17. The nucleic acid of claim 15, wherein the viral polypeptide is a viral polypeptide encoded by one of the following viruses: human immunodeficiency virus, hepatitis B virus, hepatitis C virus, hepatitis A virus, smallpox, influenza viruses, human papilloma viruses, adenoviruses, rhinoviruses, coronaviruses, herpes simplex virus, respiratory syncytial viruses, rabies, and coxsackie virus.

18. The nucleic acid of claim 15, wherein the viral polypeptide is chosen from the group consisting of the following: influenza antigens such as haemagglutinin (HA), nucleoprotein (NP), matrix protein (MP1); HIV antigens such as HIV gag, pol, env, tat, reverse transcriptase hepatitis viral antigens such as the S. M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, and other Japanese encephalitis viral antigen components; rabies viral antigens such as rabies glycoprotein, rabies nucleoprotein and other rabies viral antigen components; and Hepatitis B surface antigen.

19. The nucleic acid of any of claim 1, wherein the heterologous nucleic acid sequence encodes a mammalian polypeptide.

20. The nucleic acid of claim 19, wherein the mammalian polypeptide is a cancer-associated polypeptide or an antigenic fragment thereof.

21. The nucleic acid of claim 19, wherein the cancer-associated polypeptide is chosen from the group consisting of: 707 alanine proline (707-AP); alpha (α)-fetoprotein (AFP); adenocarcinoma antigen recognized by T cells 4 (ART-4); B antigen (BAGE); β-catenin/mutated(b-catenin/m); breakpoint cluster region-Abelson (Bcr-abl); CTL-recognized antigen on melanoma (CAMEL); carcinoembryonic antigen peptide-1 (CAP-1); caspase-8 (CASP-8); cell-division cycle 27 mutated (CDC27m); cycline-dependent kinase 4 mutated CDK4/m); carcinoembryonic antigen (CEA); cancer/testis (CT) antigen; cyclophilin B (Cyp-B); differentiation antigen melanoma (DAM-6, also known as MAGE-B2, and DAM-10, also known as MAGE-B1); elongation factor 2 mutated (ELF2M); Ets variant gene 6/acute myeloid leukemia 1 gene ETS (ETV6-AML1); glycoprotein 250 (G250); G antigen (GAGE); N-acetylglucosaminyltransferase V (GnT-V); glycoprotein 100 kD (GnT-V); helicase antigen (HAGE); human epidermal receptor-2/neurological (HER-2/neu); HLA-A*0201-R1701 (HLA-A*0201 having an arginine (R) to isoleucine (I) exchange at residue 170 of the α-helix of the α2-domain in the HLA-A2 gene); human papilloma virus E7 (HPV-E7); human papilloma virus E6 (HPV-E6); heat shock protein 70-2 mutated (HSP70-2M); human signet ring tumor-2 (HST-2); human telomerase reverse transcriptase (hTERT or hTRT); intestinal carboxyl esterase (iCE); KIAA0205; L antigen (LAGE); low density lipid receptor/GDP-L-fucose: β-D-galactosidase 2-α-Lfucosyltransferase (LDLR/FUT); melanoma antigen (MAGE); melanoma antigen recognized by T cells-1/Melanoma antigen A (MART-1/Melan-A); melanocortin 1 receptor (MC1R); myosin mutated (Myosin/m); mucin 1 (MUC 1); melanoma ubiquitous mutated 1 (MUM-1), melanoma ubiquitous mutated 2 (MUM-2), melanoma ubiquitous mutated 3 (MUM-3); New York-esophageous 1 (NY-ESO-1); protein 15 (P15); protein of 190 KD bcr-abl (p190 minor bcr-abl); promyelocytic leukaemia/retinoic acid receptor α (Pml/RARa); preferentially expressed antigen of melanoma (PRAME); prostate-specific antigen (PSA); prostate-specific membrane antigen (PSM); renal antigen (RAGE); renal ubiquitous 1 (RU1), renal ubiquitous 2 (RU2); sarcoma antigen (SAGE); SART-1; SART-3; translocation Ets-family leukemia/acute myeloid leukemia 1 (TEL/AML1); triosephosphate isomerase mutated (TPI/m); tyrosinase related protein 1 (TRP-1 or gp75); tyrosinase related protein 2 (TRP2); TRP-2/intron 2 (TRP-2/INT2); Wilms' tumor gene (WT-1).

22. The nucleic acid of claim 1, wherein the heterologous nucleic acid sequence encodes a bacterial polypeptide or an antigenic fragment thereof.

23. The nucleic acid of claim 22, wherein the bacterial polypeptide is a bacterial polypeptide encoded by one of the following bacteria: Mycobacterium spp. (e.g., Mycobacterium tuberculosis, Mycobacterium leprae), Streptococcus spp. (e.g., Streptococcus pneumoniae, Streptococcus pyogenes), Staphylococcus spp. (e.g., Staphylococcus aureus), Treponema (e.g., Treponema pallidum), Chlamydia spp., Vibrio spp. (e.g., Vibrio cholerae), Bacillus spp. (e.g., Bacillus subtilis, Bacillus anthracis), Yersinia spp. (e.g., Yersinia pestis), Neisseria spp. (e.g., Neisseria meningitides, Neisseria gonorrhoeae), Legionella spp., Bordetella spp. (e.g., Bordetella pertussis), Shigella spp., Campylobacter spp., Pseudomonas spp. (e.g., Pseudomonas aeruginosa), Brucella spp., Clostridium spp. (e.g., Clostridium tetani, Clostridium botulinum, Clostridium perfringens), Salmonella spp. (e.g., Salmonella typhi), Borrelia spp. (e.g., Borrelia burgdorferi), Rickettsia spp. (e.g., Rickettsia prowazeki), Mycoplasma spp. (e.g., Mycoplasma pneumoniae), Haemophilus spp. (e.g., Haemophilus influenzae), Branhamella spp. (e.g., Branhamella catarrhalis), Corynebacteria spp. (e.g., Corynebacteria diphtheriae), Klebsiella spp. (e.g., Klebsiella pneumoniae), Escherichia spp. (e.g., Escherichia coli), and Listeria spp. (e.g., Listeria monocytogenes).

24. The nucleic acid of claim 22, wherein the bacterial polypeptide is chosen from the group consisting of: listeriolysin O, L. monocytogenes p60, L. monocytogenes metalloprotease (MPL), Chlamydia Cap1, Chlamydia Cap2, M. tuberculosis heat shock protein (hsp)60, M. tuberculosis hsp70, M. tuberculosis Ag85, M. tuberculosis ESAT-6 and M. tuberculosis CFP10.

25. The nucleic acid of claim 1, wherein the heterologous nucleic acid sequence encodes a parasitic or fungal polypeptide.

26. The nucleic acid of claim 25, wherein the parasitic or fungal polypeptide is a polypeptide encoded by one of the following parasites or fungi: Candida spp. (e.g., Candida albicans), Cryptococcus spp. (e.g., Cryptococcus neoformans), Aspergillus spp., Histoplasma spp. (e.g., Histoplasma capsulatum), Coccidioides spp. (e.g., Coccidioides immitis), Pneumocystis (e.g., Pneumocystis carinii), Entamoeba spp. (e.g., Entamoeba histolytica), Giardia spp., Leishmania spp., Plasmodium spp., Trypanosoma spp., Toxoplasma spp. (e.g., Toxoplasma gondii), Cryptosporidium spp., Trichuris spp. (e.g., Trichuris trichiura), Trichinella spp. (e.g., Trichinella spiralis), Enterobius spp. (e.g., Enterobius vermicularis), Ascaris spp. (e.g., Ascaris lumbricoides), Ancylostoma spp., Stongyloides spp., Filaria spp., and Schistosoma spp.

27. The nucleic acid of claim 25, wherein the parasitic polypeptide is chosen from the group consisting of: MSP-1; malarial antigens 41-3, AMA-1, CSP, PFEMP-1, GBP-130, MSP-1, PFS-16, SERP; fungal antigens such as heat shock protein 60; plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

28. The nucleic acid of claim 1, wherein the Listeria monocytogenes 5′ UTR increases expression of a polypeptide encoded by the heterologous nucleic acid sequence at least 1.5-fold, 2-fold, 5-fold, 10-fold, 30-fold, or 50-fold relative to a polypeptide encoded by the heterologous nucleic acid sequence that is not operably linked to the UTR.

29. An isolated nucleic acid consisting of a Listeria monocytogenes 5′ untranslated region (UTR) selected from the group consisting of a Listeria monocytogenes hly 5′ UTR and a Listeria monocytogenes actA 5′ UTR, and functional fragments and variants thereof.

30. The nucleic acid of claim 29, wherein the Listeria monocytogenes 5′ UTR is the hly 5′ UTR or a functional fragment or variant thereof.

31. The nucleic acid of claim 29, wherein the Listeria monocytogenes 5′ UTR is the actA 5′ UTR or a functional fragment or variant thereof.

32. A nucleic acid vector comprising: a Listeria monocytogenes promoter; a Listeria monocytogenes hly 5′ untranslated region (UTR), wherein the UTR comprises a ribosome binding site; a heterologous nucleic acid sequence; a selectable marker, and a bacterial origin of replication, wherein the UTR is operably linked to the promoter and the heterologous nucleic acid sequence.

33. A nucleic acid vector comprising: a Listeria monocytogenes promoter; a Listeria monocytogenes actA 5′ untranslated region (UTR), wherein the UTR comprises a ribosome binding site; a heterologous nucleic acid sequence; a selectable marker, and a bacterial origin of replication, wherein the UTR is operably linked to the promoter and the heterologous nucleic acid sequence.

34. A bacterium comprising: a nucleic acid which comprises a promoter; a 5′ UTR, wherein the 5′ UTR comprises a Listeria manocytogenes 5′ UTR, and a ribosome binding site; and a heterologous nucleic acid sequence; wherein the UTR is operably linked to the promoter, and the heterologous nucleic acid sequence, and wherein the Listeria monocytogenes 5′ untranslated region (UTR) is selected from the group consisting of a Listeria monocytogenes hly 5′ UTR, a Listeria monocytogenes actA 5′ UTR, and functional fragments and variants thereof.

35. The bacterium of claim 34, wherein the bacterium is selected from the group consisting of:
a Listeria monocytogenes bacterium, a Bacillus subtilis bacterium, and a Lactococcus lactis bacterium.

36. A vaccine comprising a bacterium according to claim 34.

37. A vaccine comprising a nucleic acid according to claim 1.

38. A method for introducing an antigen into a eukaryotic cell, the method comprising: contacting the cells with a bacterium, wherein the bacterium comprises a nucleic acid comprising: a promoter; a 5′ UTR, wherein the 5′ UTR comprises a Listeria monocytogenes 5′ UTR and an RBS; and a heterologous nucleic acid sequenceUTR, wherein the UTR is operably linked to the RBS, and the heterologous nucleic acid sequence, and wherein the Listeria monocytogenes 5′ untranslated region (UTR) is selected from the group consisting of a Listeria monocytogenes hly 5′ UTR, a Listeria monocytogenes actA 5′ UTR, a Listeria monocytogenes hly 5′ UTR and a Listeria monocytogenes actA 5′ UTR, and functional fragments and variants thereof.

39. A method for inducing an immune response to an antigen in a subject, the method comprising:
administering to the subject a plurality of bacteria, wherein each bacterium comprises a nucleic acid, comprising: a promoter, a 5′ UTR, wherein the 5′ UTR comprises a Listeria monocytogenes 5′ UTR and an RBS; and a heterologous nucleic acid sequence, wherein the UTR is operably linked to the RBS, and the heterologous nucleic acid sequence, and wherein the Listeria monocytogenes 5′ untranslated region (UTR) is selected from the group consisting of a Listeria monocytogenes hly 5′ UTR, a Listeria monocytogenes actA 5′ UTR, a Listeria monocytogenes hly 5′ UTR and a Listeria monocytogenes actA 5′ UTR, and functional fragments and variants thereof.

40. A method for expressing a polypeptide, the method comprising:
introducing into a bacterium a nucleic acid according to claim 1, wherein the heterologous nucleic acid sequence encodes a polypeptide, and expressing the polypeptide.
